(19) United States
(12) Patent Application Publication
(10) Pub. No.: US 2023/0346318 A1
(43) Pub. Date: Nov. 2, 2023

(54) INFORMATION PROCESSING APPARATUS, INFORMATION PROCESSING METHOD, AND NON-TRANSITORY COMPUTER READABLE MEDIUM

(71) Applicants: NEC Corporation, Tokyo (JP); NATIONAL UNIVERSITY CORPORATION TOKYO MEDICAL AND DENTAL UNIVERSITY, Tokyo (JP)

(72) Inventors: Makoto YASUKAWA, Tokyo (JP); Shuhei NOYORI, Tokyo (JP); Kosuke Nishihara, Tokyo (JP); Yuki Kosaka, Tokyo (JP); Akimoto Nimura, Tokyo (JP); Koji Fujita, Tokyo (JP); Takuya Ibara, Tokyo (JP)

(73) Assignees: NEC Corporation, Tokyo (JP); NATIONAL UNIVERSITY CORPORATION TOKYO MEDICAL AND DENTAL UNIVERSITY, Tokyo (JP)

(21) Appl. No.: 18/128,561

(22) Filed: Mar. 30, 2023

(30) Foreign Application Priority Data
Mar. 31, 2022 (JP) 2022-058199

(51) Int. Cl. A61B 5/00; A61B 5/08

(52) U.S. Cl. CPC A61B 5/7235; A61B 5/0803; A61B 5/7275

(57) ABSTRACT

An information processing apparatus inputs expiratory phase data being data of expiratory phase and inspiratory phase data being data of inspiratory phase, for each of abdominal waveform data indicating a breathing waveform of an abdomen of a subject performing breathing training and chest waveform data indicating a breathing waveform of a chest of the subject. The information processing apparatus calculates a phase difference between the abdominal waveform data and the chest waveform data in each of the expiratory phase and the inspiratory phase.

### INCORPORATION BY REFERENCE

This application is based upon and claims the benefit of priority from Japanese patent application No. 2022-058199, filed on Mar. 31, 2022, the disclosure of which is incorporated herein in its entirety by reference.

### TECHNICAL FIELD

The present disclosure relates to an information processing apparatus, an information processing method, and a program.

### BACKGROUND ART

For improving and maintaining a health condition, it is desirable to breathe by a correct breathing method. In order to confirm whether breathing is performed by the correct breathing method, movements of a chest portion and an abdomen portion are detected. In this regard, Japanese Unexamined Patent Application Publication No. 2008-154655 discloses a technique of measuring a movement of a chest portion and a movement of an abdomen portion of a patient who breathes, from an image acquired by photographing the chest portion and the abdomen portion by using a pattern light projection apparatus and a camera.

In a breathing training by the correct breathing method, it is important that a movement of a chest portion of a subject performing training in a front-rear direction and a movement of an abdomen portion of the subject in a front-rear direction are synchronized with each other. In addition, it is desirable that a doctor and the like or the subject himself/herself can easily recognize whether the correct breathing is performed with such synchronization, and in particular, it is desirable to be able to recognize at which timing the synchronization is not achieved.

However, in the technique described in Japanese Unexamined Patent Application Publication No. 2008-154655, it is only possible to detect a switching time of an expiratory phase and an inspiratory phase from a peak of a chest and abdominal waveform and acquire a delay time thereof, and it is not possible to confirm synchrony between the abdominal waveform and the chest waveform in each phase.

An example object of the present disclosure is to provide an information processing apparatus, an information processing method, and a program that are capable of acquiring information indicating at which timing synchronization between an abdomen and a chest of a subject performing breathing training is not achieved.

### SUMMARY

In a first example aspect, an information processing apparatus according to the present disclosure includes: an input unit configured to input expiratory phase data being data of expiratory phase and inspiratory phase data being data of inspiratory phase, for each of abdominal waveform data indicating a breathing waveform of an abdomen of a subject performing breathing training and chest waveform data indicating a breathing waveform of a chest of the subject; and a calculation unit configured to calculate a phase difference between the abdominal waveform data and the chest waveform data in each of the expiratory phase and the inspiratory phase.

In a second example aspect, an information processing method according to the present disclosure includes: inputting expiratory phase data being data of expiratory phase and inspiratory phase data being data of inspiratory phase, for each of abdominal waveform data indicating a breathing waveform of an abdomen of a subject performing breathing training and chest waveform data indicating a breathing waveform of a chest of the subject; and calculating a phase difference between the abdominal waveform data and the chest waveform data in each of the expiratory phase and the inspiratory phase.

In a third example aspect, a program according to the present disclosure is a program that causes a computer to execute information processing including: inputting expiratory phase data being data of expiratory phase and inspiratory phase data being data of inspiratory phase, for each of abdominal waveform data indicating a breathing waveform of an abdomen of a subject performing breathing training and chest waveform data indicating a breathing waveform of a chest of the subject; and calculating a phase difference between the abdominal waveform data and the chest waveform data in each of the expiratory phase and the inspiratory phase.

### BRIEF DESCRIPTION OF DRAWINGS

The above and other aspects, features and advantages of the present disclosure will become more apparent from the following description of certain example embodiments when taken in conjunction with the accompanying drawings, in which:

FIG. 1 is a block diagram illustrating a configuration example of an information processing apparatus according to a first example embodiment;

FIG. 2 is a flowchart for explaining an example of an information processing method in the information processing apparatus in FIG. 1;

FIG. 3 is a block diagram illustrating a configuration example of a display system including the information processing apparatus according to a second example embodiment;

FIG. 4 is a schematic side view illustrating an appearance of the display system in FIG. 3;

FIG. 5 is a schematic diagram illustrating an example of a photographed image acquired by an imaging device in the display system in FIG. 3;

FIG. 6 is a graph illustrating an example of chest waveform data and abdominal waveform data acquired by the information processing apparatus in the display system in FIG. 3;

FIG. 7 is a diagram illustrating a top view schematically illustrating an example of a band-type waveform acquisition sensor that can be adopted in place of the imaging device in the display system in FIGS. 3 and 4;

FIG. 8 is a diagram illustrating an example of a distribution of phase differences displayed on a display device under control of an information processing apparatus in the display system in FIG. 3;

FIG. 9 is a diagram illustrating another example of a distribution of phase differences displayed on the display device under the control of the information processing apparatus in the display system in FIG. 3;

FIG. 10 is a diagram illustrating an example of an image including a distribution of phase differences displayed on a display device under the control of an information processing apparatus in the display system of FIG. 3;

FIG. 11 is a graph illustrating an example of chest waveform data and abdominal waveform data of data 1 in FIG. 10;

FIG. 12 is a graph illustrating an example of chest waveform data and abdominal waveform data of data 2 in FIG. 10;

FIG. 13 is a diagram illustrating another example of an image including a distribution of phase differences displayed on a display device under the control of an information processing apparatus in the display system in FIG. 3;

FIG. 14 is a diagram illustrating another example of the image including the distribution of phase differences displayed on the display device under the control of the information processing apparatus in the display system in FIG. 3;

FIG. 15 is a flowchart for explaining an example of processing in the display system in FIG. 3;

FIG. 16 is a block diagram illustrating a configuration example of a display system including the information processing apparatus according to a third example embodiment;

FIG. 17 is a diagram illustrating an example of a transition of displacement displayed on the display device under the control of the information processing apparatus in the display system in FIG. 3;

FIG. 18 is a diagram illustrating another example of the transition of the displacement displayed on the display device under the control of the information processing device in the display system of FIG. 3;

FIG. 19 is a flowchart for explaining an example of processing in the display system in FIG. 16; and

FIG. 20 is a diagram illustrating an example of a hardware configuration included in the apparatus.

### EXAMPLE EMBODIMENT

Hereinafter, example embodiments will be explained with reference to the drawings. For clarity of explanation, the following description and the drawings are omitted and simplified as appropriate. In the drawings, the same elements are denoted by the same reference numerals, and redundant explanations are omitted as necessary.

### First Example Embodiment

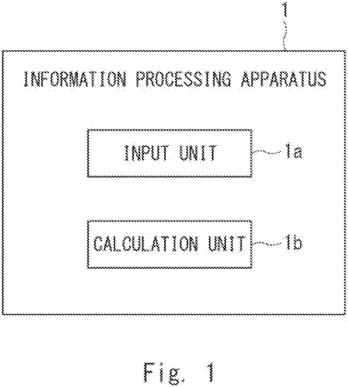

FIG. 1 is a block diagram illustrating a configuration example of an information processing apparatus 1 according to a first example embodiment. As illustrated in FIG. 1, the information processing apparatus 1 may include an input unit 1a and a calculation unit 1b, and may be used for breathing training.

In order to improve and maintain a health condition, it is desirable to perform breathing with a correct breathing method, and in order to perform the correct breathing, it is desirable to continuously perform correct breathing training based on a guidance of a training instructor such as a doctor or a therapist (hereinafter, simply referred to as “instructor”). For example, breathing training with the correct breathing method may improve health conditions such as a physical function, such as a low back pain, and a mental status.

Herein, in the breathing training, it is considered that an effect of the training is better by the subject breathing in such a way that anteroposterior movements of a chest portion and an abdomen portion are synchronized with each other (“synchronization between the chest portion and the abdomen portion”). Furthermore, it is considered that the training effect is better by the subject breathing in such a way as to satisfy that ribs are sufficiently internally rotated when exhaling (at exhalation) (i.e., that the displacement amount of the chest portion in a left-right direction becomes sufficiently small during exhalation; “internal rotation of ribs”). However, it is difficult for the subject himself/herself to confirm the above. In other words, it is difficult for the subject to recognize his/her own breathing state.

In order to enable such recognition, the information processing apparatus 10 according to the present example embodiment is used. Components of the information processing apparatus 10 will be explained.

The input unit 1a is a part that inputs expiratory phase data and inspiratory phase data and passes them to the calculation unit 1b, and it is possible to include an interface for that purpose.

The expiratory phase data being input by the input unit 1a are data of expiratory phase for each of abdominal waveform data indicating a breathing waveform of an abdomen portion of a subject and chest waveform data indicating a breathing waveform of a chest portion of the subject. Herein, the subject is a person who performs breathing training. The inspiratory phase data being input by the input unit 1a are data of inspiratory phase for each of the abdominal waveform data and the chest waveform data.

Note that the expiratory phase data and the inspiratory phase data may be, for example, a series of data as data, and in this case, any data capable of identifying the expiratory phase and the inspiratory phase may be used. For example, information capable of identifying the expiratory phase and the inspiratory phase may be added in the abdominal waveform data, and information capable of identifying the expiratory phase and the inspiratory phase may be added in the chest waveform data.

The calculation unit 1b calculates a phase difference between the abdominal waveform data and the chest waveform data in each of the expiratory phase and the inspiratory phase. The calculation unit 1b may calculate at least one phase difference for the expiratory phase and calculate at least one phase difference for the inspiratory phase. An example of calculating a plurality of phase differences for each phase will be explained in a second example embodiment.

Accordingly, the phase difference between the abdominal waveform data and the chest waveform data in the expiratory phase of the subject is calculated, and the phase difference between the abdominal waveform data and the chest waveform data in the inspiratory phase of the subject is calculated, and the phase difference in each phase can be acquired. By acquiring the phase difference in each phase, it is possible to know which phase is not synchronized in either the expiratory phase or the inspiratory phase, which phase is not synchronized, and the like.

Such information can be used as information for supporting a breathing training. Therefore, the information processing apparatus 1 can be referred to as a breathing training support apparatus. The breathing training is also referred to as a breathing exercise practice.

The information processing apparatus 1 illustrated in FIG. 1 may be, for example, a computer such as a server or a personal computer, or may be an apparatus including dedicated hardware. Specifically, the information processing apparatus 1 may include a computer apparatus including hardware including, for example, one or more processors and one or more memories. At least a part of functions of each unit in the information processing apparatus 1 may be achieved by one or more processors operating in accordance with a program read from one or more memories.

In other words, the information processing apparatus 1 may include a control unit (not illustrated) that controls the whole of the information processing apparatus. The control unit can be achieved by, for example, a central processing unit (CPU) or a graphics processing unit (GPU), a working memory, a non-volatile storage device storing a program, and the like. This program can be a program for causing the CPU or the GPU to execute processing of the input unit 1a and the calculation unit 1b.

Further, the information processing apparatus 1 may include a storage device that stores the input expiratory phase data and inspiratory phase data, and the calculated phase difference, and as the storage device, a storage device included in the control unit may be used for example.

Further, the information processing apparatus 1 is not limited to an example configured as a single apparatus, and may be constructed as a plurality of apparatuses in which functions are distributed, i.e., as an information processing system, and a method of distribution thereof is not limited. In a case of constructing an information processing system in which functions are distributed among a plurality of apparatuses, each apparatus may be provided with a control unit, a communication unit, and as necessary, a storage unit and the like and the plurality of apparatuses may be connected as necessary by wireless or wired communication and may achieve the functions explained in the information processing apparatus 1 in cooperation with each other.

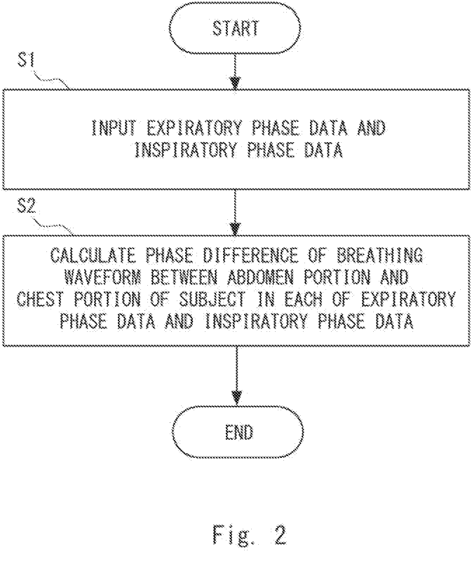

Next, a processing example of the information processing apparatus 1 will be explained with reference to FIG. 2. FIG. 2 is a flowchart for explaining an example of an information processing method in the information processing apparatus 1 in FIG. 1.

First, the information processing apparatus 1 inputs the expiratory phase data and the inspiratory phase data for each of the abdominal waveform data and the chest waveform data for the subject performing the breathing training (step S1). Next, the information processing apparatus 1 calculates a phase difference between the abdominal waveform data and the chest waveform data in each of the expiratory phase and the inspiratory phase (step S2), and ends the processing.

As described above, in the present example embodiment, the phase difference between the abdominal waveform data and the chest waveform data of the subject performing the breathing training can be acquired in the expiratory phase and the inspiratory phase. Therefore, according to the present example embodiment, it is possible to acquire information on synchrony between the chest portion and the abdomen portion during breathing, which is important in breathing training, at least in a state comparable between the expiratory phase and the inspiratory phase. In short, according to the present example embodiment, it is possible to acquire information for quantitatively evaluating the synchrony between the chest and abdomen portions during the breathing of the subject by dividing the synchrony into the expiratory phase and the inspiratory phase, and thereby it is possible to quantitatively evaluate the synchrony.

As described above, according to the present example embodiment, it is possible to acquire information indicating at which timing synchronization between the abdomen portion and the chest portion of the subject performing the breathing training is not achieved. In addition, this enables timely feedback of this information to the subject or an instructor who sends advice to the subject, thereby enabling more effective breathing training to be performed.

In addition, in the present example embodiment, at a time of rehabilitation in a medical institution or breathing exercise practice in a healthcare service, effective guidance can be provided by use of an instructor such as a therapist. Further, by mounting the information processing apparatus 1 on a terminal device or the like to be used by the subject, the subject can receive remote instruction from the instructor or perform voluntary training while being at home.

### Second Example Embodiment

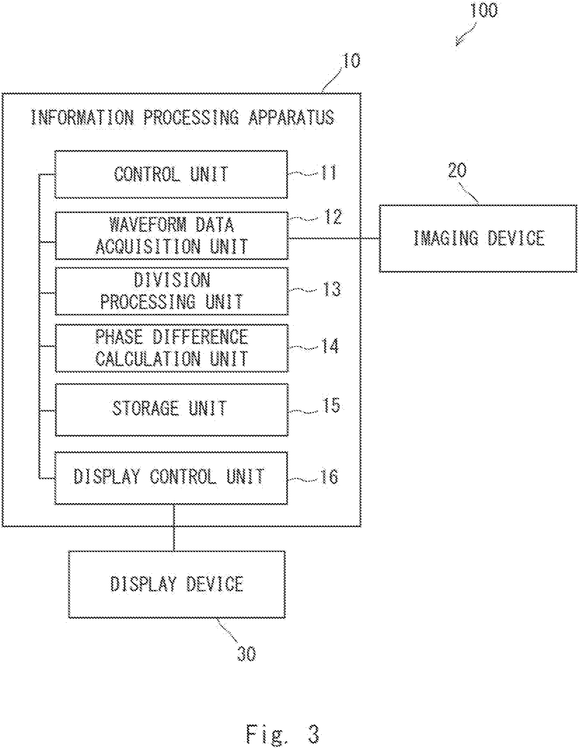

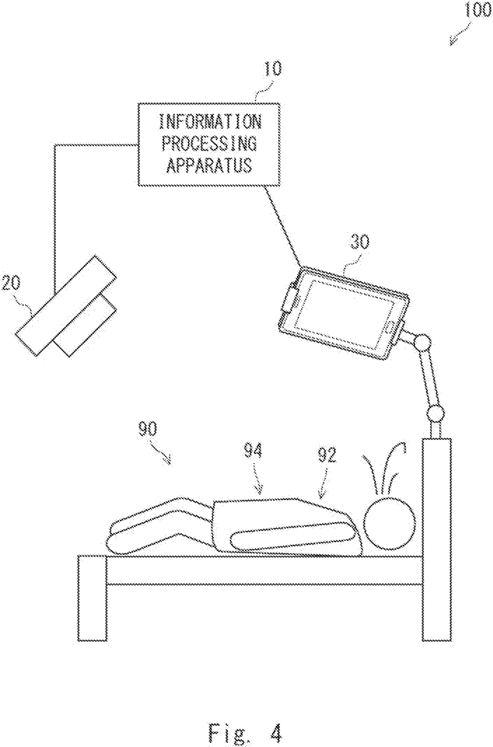

Although the second example embodiment will be mainly explained with reference to FIGS. 3 to 15, various examples explained in the first example embodiment can be applied. First, a configuration example of an information display system (hereinafter, simply referred to as a display system) including the information processing apparatus according to the present example embodiment will be explained with reference to FIGS. 3 and 4. FIG. 3 is a block diagram illustrating a configuration example of a display system including an information processing apparatus according to the second example embodiment, and FIG. 4 is a schematic side view illustrating an external appearance of the display system.

As illustrated in FIGS. 3 and 4, a display system 100 according to the present example embodiment includes an information processing apparatus 10 that is an example of the information processing apparatus 1 in FIG. 1, at least one imaging device 20, and at least one display device 30. The information processing apparatus 10 is communicably connected to the imaging device 20 and the display device 30 via a wired or wireless network.

As illustrated in FIG. 4, a display system 100 can be used for breathing training of a subject 90. As illustrated in FIG. 4, the subject 90 can perform breathing training in a supine (supine position) state, but a posture of the subject 90 is not limited to the supine position. However, in the following explanation, it is assumed that the subject 90 performs the breathing training in the supine position, and examples of other postures will be supplementarily explained.

The imaging device 20 photographs the subject 90 who performs breathing training. The imaging device 20 may be installed at a position where images of a chest portion 92 and an abdomen portion 94 of the subject 90 can be photographed. When the subject 90 performs breathing training in a supine position, the imaging device 20 may be installed on an upper side of the chest portion 92 and the abdomen portion 94 of the subject 90, for example, as illustrated in FIG. 4. In other words, the imaging device 20 can be installed at a position facing the subject 90 in the supine position. Note that the subject 90 may perform breathing training in a state of wearing clothes. In this case, the chest portion 92 is a portion associated to the chest portion of the subject 90 in a state of wearing clothes. Similarly, the abdomen portion 94 is a portion associated to the abdomen portion of the subject 90 in a state of wearing clothes.

The imaging device 20 is, for example, a camera. The imaging device 20 may be a two-dimensional camera (e.g., an RGB camera, etc.), a three-dimensional camera, or a camera including both of them (e.g., an RGB-D camera, etc.). Examples of the three-dimensional cameras include a depth sensor, Light Detection and Ranging (LiDAR), a stereo camera, and the like. The imaging device 20 may measure a distance to a captured object by, for example, a Time of Flight (ToF) method.

By using the imaging device 20, it is possible to detect a position of the subject 90 and a movement that is a change thereof. For example, motion capture or the like can be achieved by using the imaging device 20. Furthermore, skeletal data indicating a skeleton (joint) of the subject 90 that has been photographed may be generated by using the imaging device 20. The skeleton data are data indicating a position of the joint of the subject 90. The skeleton data can be acquired, for example, by the imaging device 20 (or the information processing apparatus 10) recognizing the joint of a moving person.

The imaging device 20 generates image data indicating at least the chest portion 92 and the abdomen portion 94 of the subject 90 by photographing the subject 90. In short, the image data may indicate the chest portion 92 and abdomen portion 94 of the subject 90 and the images (photographed images) around them. The photographed image may be a moving image or a still image. In the following description, the term “image” also means “image data indicating an image” as a processing target in information processing.

Further, as described above, the image data may be, for example, two-dimensional image data such as an RGB image, or three-dimensional image data such as a distance image represented by three-dimensional point cloud data. Alternatively, the image data may be data indicating an image acquired by combining a two-dimensional image and a three-dimensional image. Accordingly, the image data may indicate position information on a position of the surface of the photographed subject 90 as three-dimensional coordinates, by the three-dimensional point cloud data or the like. The image data may include the skeleton data described above. The imaging apparatus 20 transmits the generated image data to the information processing apparatus 10. By using the image data, a position of the chest portion 92 and a position of the abdomen portion 94 of the subject 90 can be detected. In addition, it is possible to detect data indicating a change in the positions by time-series image data. In short, movement of the chest portion 92 and movement of the abdomen portion 94 of the subject 90 can be detected by using the time-series image data. The position of the detection target includes at least a position in a vertical direction in the supine position, which is the position in the up-down direction in FIG. 4, but may also include a position in a horizontal direction in the supine position. As described above, the imaging device 20 can also function as a detection device capable of detecting the position of the subject 90 and the movement that is a change thereof, and outputting each of the chest waveform data that are data indicating a change in the position of the chest portion 92 and the abdominal waveform data that are data indicating a change in the position of the abdomen portion 94.

In the following explanation, for convenience it is assumed that the imaging device 20 outputs image data to the information processing apparatus 10, and the information processing apparatus 10 detects the position of the chest portion 92 and the position of the abdomen portion 94 from the image data, acquiring the chest waveform data and the abdominal waveform data. However, it is also possible to adopt a configuration in which the imaging device 20 has a function as a detection device and outputs the chest waveform data and the abdominal waveform data to the information processing apparatus 10.

The information processing apparatus 10 acquires image data from the imaging device 20. The information processing apparatus 10 analyzes the acquired image data, thereby detecting each of the displacement amounts of the chest portion 92 and the abdomen portion 94 of the subject 90. The detected displacement amount is acquired as time-series chest waveform data and abdominal waveform data indicating the change. The detected displacement amount includes at least the displacement amount in the vertical direction in the supine position as described above, but may also include the displacement amount in the horizontal direction in the supine position. The following will be explained on the assumption that the displacement amount refers to the displacement amount in the vertical direction in the supine position, and the displacement amount in the horizontal direction will be described as necessary.

The information processing apparatus 10 performs control of calculating a phase difference between the abdominal waveform data and the chest waveform data in each of the expiratory phase and the inspiratory phase on the basis of the chest waveform data and the abdominal waveform data indicating each of the detected displacement amounts of the chest portion 92 and the abdomen portion 94, and to display the calculated phase difference on the display device 30. In short, the display device 30 can display information indicating the phase difference between the abdominal waveform data and the chest waveform data in the expiratory phase of the subject 90 and the phase difference between the abdominal waveform data and the chest waveform data in the inspiratory phase of the subject 90 under the control of the information processing apparatus 10. This will be described later with reference to specific examples.

In addition, the information processing apparatus 10 can also perform control of displaying, on the display device 30, information related to the training of the subject 90 other than the information indicating the phase differences. In short, the display device 30 can display information related to the training of the subject 90 other than the information indicating the phase differences under the control of the information processing apparatus 10.

Further, in the display device 30, the information can be displayed as information for the subject 90 or as information for an instructor, and the display contents can be made different in consideration of clarity or the like depending on a target person of browsing. For example, when the display device 30 is a display device possessed by the instructor, the information for the instructor can be displayed. Further, as illustrated in FIG. 4, the display device 30 may display an image for the subject 90 when the display device 30 is installed over the head of the subject 90. For example, the display device 30 may display an image for the subject 90 when the camera built in the display device 30 detects a face of the subject 90.

Although the following will be explained on the assumption that the display device 30 is used for the subject 90 to browse the information, the display device 30 may be used for the instructor to browse the information, or a plurality of display devices 30 for the subject 90 and the instructor may be provided in the display system 100.

The display device 30 is arranged in such a way as to display an image at a position visible from the subject 90. The display device 30 includes, for example, a display for displaying an image. The display device 30 includes, for example, a Liquid Crystal Display (LCD), but is not limited thereto. The display device 30 may be achieved by an organic Electro-Luminescence (EL) display, a projector, or the like. The display device 30 may be, for example, a smartphone, a tablet terminal, or the like. Details of the contents displayed by the display device 30 will be described later.

Next, a specific configuration example of the information processing apparatus 10 will be explained. As illustrated in FIG. 3, the information processing apparatus 10 may include a control unit 11, a waveform data acquisition unit 12, a division processing unit 13, a phase difference calculation unit 14, a storage unit 15, and a display control unit 16.

The control unit 11 is a part that controls the entire information processing apparatus 10, and may include a processor such as a CPU or a GPU. The control unit 11 has a function as an arithmetic device that performs control processing, arithmetic processing, and the like.

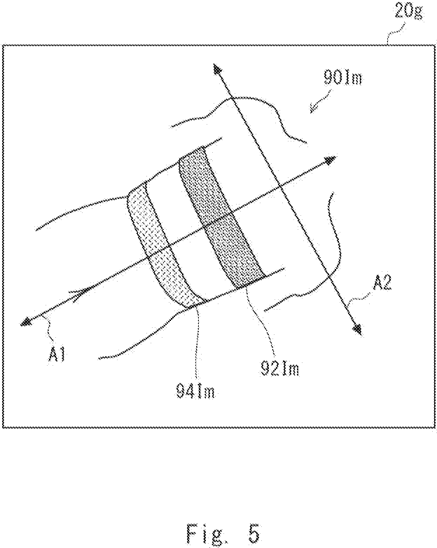

The waveform data acquisition unit 12 may include an interface such as a communication interface for wired or wireless connection to the imaging device 20. Then, the waveform data acquisition unit 12 inputs the image data from the imaging device 20 at predetermined time intervals, for example, analyzes the image data, and detects each of the displacement amounts of the chest portion 92 and the abdomen portion 94 of the subject 90. An example of detection of the displacement amount will be described later with reference to FIG. 5. The displacement amount of the detection target can be a displacement amount of the subject 90 in at least one direction in a front-rear direction (vertical direction in the supine position), a left-right direction, and an up-down direction (direction from the top of the head toward the foot).

The waveform data acquisition unit 12 can acquire time-series chest waveform data indicating a change as the displacement amount of the chest portion 92 and time-series abdominal waveform data indicating a change as the displacement amount of the abdomen portion 94, and the acquired chest waveform data and the abdominal waveform data are passed to the division processing unit 13. In this way, the waveform data acquisition unit 12 is an example of a waveform data input unit that inputs the abdominal waveform data and the chest waveform data and passes them to the division processing unit 13.

The division processing unit 13 divides each of the abdominal waveform data and the chest waveform data into expiratory phase data and inspiratory phase data, based on average waveform data of the abdominal waveform data and the chest waveform data. However, as explained in the first example embodiment, the expiratory phase data and the inspiratory phase data may be, for example, a series of data as data, and in this case, any data capable of identifying the expiratory phase and the inspiratory phase may be used. For example, information capable of identifying the expiratory phase and the inspiratory phase may be added in the abdominal waveform data, and information capable of identifying the expiratory phase and the inspiratory phase may be added in the chest waveform data. The division processing unit 13 may also be referred to as an expiratory /inspiratory phase division processing unit in order to divide the expiratory phase and the inspiratory phase.

Herein, a reason why the average waveform data are used will be explained supplementarily. When the breathing is such that the phases of the chest portion and the abdomen portion are shifted, peaks of the chest waveform data and the abdomen waveform data are shifted from each other. Therefore, when the expiratory phase and the inspiratory phase are calculated based on the peaks and the like independently of the chest waveform data and the abdominal waveform data, ratios of the expiratory phase and the inspiratory phase differ between the chest portion and the abdomen portion. In order to calculate the phase difference by comparing the phases associated to the chest waveform data and the abdominal waveform data in the subsequent phase difference calculation unit 14, the above-described ratios need to be the same. Therefore, the division processing unit 13 uses the average waveform data and determines a division position between the expiratory phase and the inspiratory phase (a position at which the phase changes during one breathing cycle).

The division processing unit 13 only needs to divide the expiratory phase and the inspiratory phase from the average waveform, and the method thereof is not limited. For example, the average waveform may be displayed on the display device 30 or the like, and a division instruction operation by the instructor may be accepted and the average waveform may be divided according to the operation. Alternatively, the division processing unit 13 may analyze the average waveform data, detect the peak, and divide the expiratory phase and the inspiratory phase according to a predetermined rule based on the detected peak. Alternatively, the division processing unit 13 may be configured to divide the expiratory phase and the inspiratory phase by using a machine learning method such as a hidden Markov model.

As described above, the division processing unit 13 divides the expiratory phase and the inspiratory phase from the average waveform data. The waveform data acquisition unit 12 and the division processing unit 13 are examples of the input unit 1a in FIG. 1. The division processing unit 13 passes the divided expiratory phase data and inspiratory phase data to the phase difference calculation unit 14.

The phase difference calculation unit 14 is an example of the calculation unit 1b in FIG. 1, and calculates a phase difference between the abdominal waveform data and the chest waveform data in the expiratory phase and a phase difference between the abdominal waveform data and the chest waveform data in the inspiratory phase. Since the phase difference calculation unit 14 detects the phase difference between the chest portion 92 and the abdomen portion 94, it may be referred to as a chest and abdomen breathing phase difference detection unit. Further, since the average waveform is divided into the expiratory phase and the inspiratory phase by the division processing unit 13, it can be said that the phase difference calculated by the phase difference calculation unit 14 is an estimated value. Therefore, the phase difference calculation unit 14 may be referred to as a phase difference estimation unit.

The method of calculating the phase difference in each phase by the phase difference calculation unit 14 is not limited. For example, the phase difference calculation unit 14 may perform Hilbert conversion on the abdominal waveform data and the chest waveform data in the expiratory phase and calculate each of successive phases (instantaneous phases) for the abdomen portion and the chest portion. Similarly, the phase difference calculation unit 14 may perform Hilbert conversion on the abdominal waveform data and the chest waveform data in the inspiratory phase and calculate each of successive phases (instantaneous phases) for the abdomen portion and the chest portion. Then, the phase difference calculation unit 14 calculates the phase difference between the instantaneous phase of the abdomen portion and the instantaneous phase of the chest portion which are calculated in this manner.

In addition, the phase difference calculation unit 14 can reduce the number of pieces of data in order to reduce a target of display control in a display control unit 16 to be described later and make it easy to visually recognize. For example, the phase difference calculation unit 14 can normalize the calculated phase difference in such a way that a length of the sequence expresses one breathing cycle at 100%, and can calculate 10 average phase differences by taking an average in units of 10%, for example. By such normalization, the breathing waveform can be divided for each breath, and the length of each breath can be unified. Of course, a target period to be averaged at this time is not limited to 10%, and the average phase difference of the number in response to the period is calculated.

In addition, in a simple example, only two values of an average phase difference in the expiratory phase and an average phase difference in the inspiratory phase may be calculated for one breathing cycle. In addition, another statistical value such as a median value may be calculated instead of the calculation of the average value, or all the phase differences at the data interval existing as it is without performing statistical processing can be also targets of display control in the display control unit 16 to be described later.

As described above, the phase difference calculation unit 14 can calculate the phase difference during the normalized breathing cycle for the inspiratory phase data and the expiratory phase data. Thus, the display control unit 16 in the subsequent stage can display a distribution of the phase difference during the normalized breathing cycle of the expiratory phase and the inspiratory phase on the display device 30.

The storage unit 15 is, for example, a storage device such as a memory or a hard disk. The storage unit 15 is, for example, a Read Only Memory (ROM), a Random Access Memory (RAM), or the like. The storage unit 15 has a function for storing a control program, an arithmetic program, and the like that are executed by the control unit 11. In addition, the storage unit 15 has a function of temporarily storing data or the like during processing, and a function of storing data after processing in order to refer to the data as information to be displayed on the display device 30 by the display control unit 16 to be described later. The storage unit 15 may have a function of storing the processed data in order refer to the data as past data to be described later. The storage unit 15 may have a function of storing processed data and the like in a database format.

The display control unit 16 may include an interface such as a communication interface for wired or wireless connection to the display device 30. The display control unit 16 controls the display device 30 to display the distribution of the phase difference calculated by the phase difference calculation unit 14.

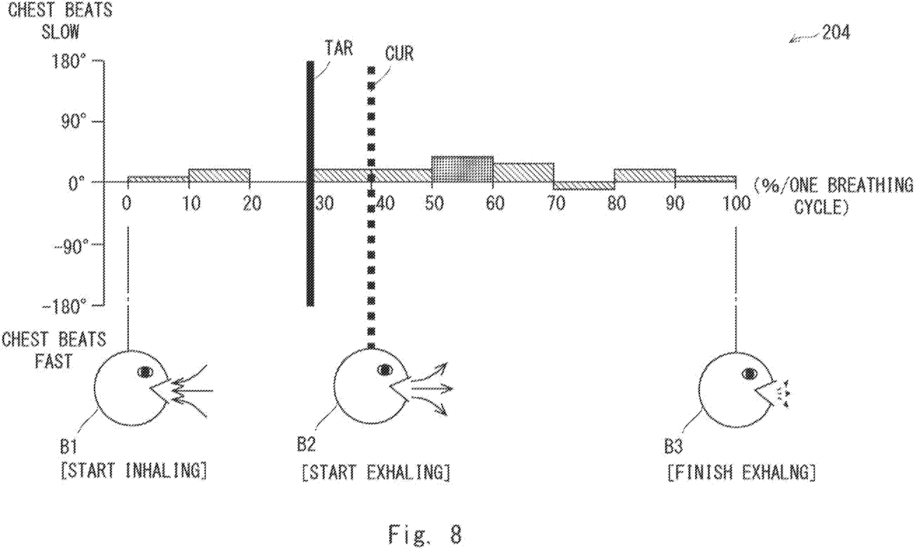

The distribution may include at least a value of a phase difference between the abdominal waveform data and the chest waveform data during one breathing cycle consisting of one expiratory phase and one inspiratory phase. In short, the distribution may include at least one set of the value of the phase difference in the expiratory phase and the value of the phase difference in the inspiratory phase which are calculated by the phase difference calculation unit 14. However, this distribution may include, for example, a value indicating a phase difference for each of a plurality of periods in which the expiratory phase is divided into, for example, equal intervals in the expiratory phase, and the same applies to the inspiratory phase. In this case, the values of the plurality of phase differences to be displayed can be calculated by the phase difference calculation unit 14. Alternatively, this distribution may include a value indicating a phase difference for each of a plurality of time periods in which one breathing cycle is divided into equal intervals. In this case as well, the values of the plurality of phase differences to be displayed can be calculated by the phase difference calculation unit 14. In this case, however, it is assumed that the division is performed in such a way that at least one value is included in each of the expiratory phase and the inspiratory phase. Examples of the display will be described later with reference to FIG. 8 and the like.

With such display control, in the display device 30, a value indicating a phase difference between the abdominal waveform data and the chest waveform data in the expiratory phase of the subject 90 and a value indicating a phase difference between the abdominal waveform data and the chest waveform data in the inspiratory phase of the subject 90 can be displayed. In particular, in the display device 30, the value in the expiratory phase and the value in the inspiratory phase can be displayed as a distribution, i.e., at least as can be seen to be the value of each phase. Therefore, by such a display, it is possible to allow the subject 90 or the like to visually recognize, for example, which of the expiratory phase and the inspiratory phase is not synchronized, or which of the phases is not synchronized.

In addition, in a case where a plurality of phase difference values are displayed in at least one of the expiratory phase and the inspiratory phase, it is possible to finely feed back to the subject 90 directly or through the instructor at which timing in the target phase the synchronization is not achieved. This allows the subject 90 to perform more effective breathing training.

In particular, by displaying a plurality of phase difference values for both phases, by further increasing the number of phase differences to be displayed, it is possible to feed back in detail information indicating at which timing the synchronization is not achieved to the subject 90 directly or via the instructor. In short, by presenting such detailed information, it is possible to promote understanding of synchrony during exhalation and during inspiration by more finely providing feedback to the subject 90 directly or through the instructor.

The components of the waveform data acquisition unit 12, the division processing unit 13, the phase difference calculation unit 14, and the display control unit 16 in the information processing apparatus 10 can be achieved by, for example, causing a program to be executed under the control of the control unit 11. More specifically, each component can be achieved by the control unit 11 executing a program stored in the storage unit 15. In addition, necessary programs may be recorded in an optional nonvolatile recording medium and installed as necessary, and thereby may achieve each component. Further, each component is not limited to being achieved by software by a program, and may be achieved by any combination and the like of hardware, firmware, and software. In addition, each component may be achieved by use of an integrated circuit, which is programmable by a user, such as a field-programmable gate array (FPGA) or a microcomputer. In this case, by use of the integrated circuit, a program composed of the above-described components may be achieved.

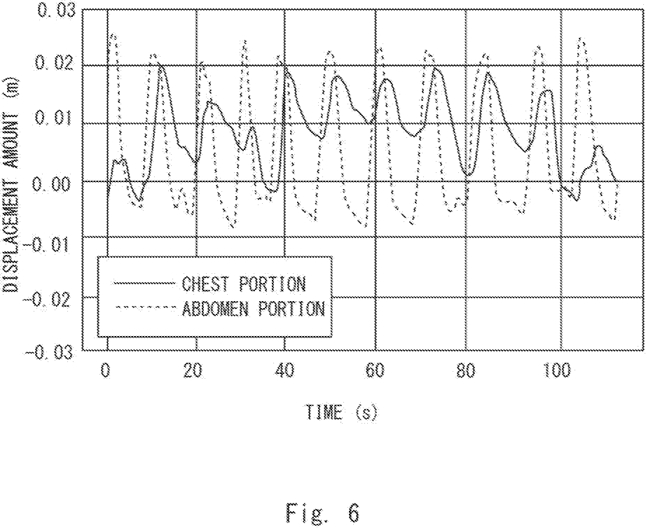

Next, with reference to FIGS. 5 and 6, an example of processing in which the waveform data acquisition unit 12 detects a displacement amount and acquires time-series chest waveform data and abdominal waveform data will be explained. FIG. 5 is a schematic diagram illustrating an example of a photographed image acquired by the imaging device 20 in FIG. 3. FIG. 6 is a graph illustrating an example of chest waveform data and abdominal waveform data acquired by the information processing apparatus 10.

A photographed image 20g illustrated in FIG. 5 is an image indicated by the image data acquired by the imaging device 20, and may include three-dimensional information. When three-dimensional information is included in the photographed image 20g, a pixel constituting the photographed image 20g may include positional information (distance information, i.e., depth information) of a portion of the subject associated to the pixel.

The imaging device 20 acquires an image of the subject 90 in a rest state and transmits the image to the waveform data acquisition unit 12. Specifically, the instructor instructs the subject 90 to relax and rest, and the imaging device 20 photographs the subject 90 in the state and transmits the photographed image to the waveform data acquisition unit 12. As a result, the waveform data acquisition unit 12 acquires image data of the subject 90 at the normal time, i.e., at the time of breathing during rest. Note that the image of the subject 90 may be a moving image or a still image.

The photographed image 20g includes a subject image 90Im that is an image of the subject 90, and the subject image 90Im includes a chest region 92Im and an abdominal region 94Im. The waveform data acquisition unit 12 can input image data indicating the photographed image 20g from the imaging device 20, analyze the image data, and specify the chest region 92Im and the abdominal region 94Im. In short, the waveform data acquisition unit 12 can have a function of specifying such a region.

This specific method is not limited. The waveform data acquisition unit 12 may specify a region associated to the chest portion 92 in the image by using, for example, skeleton data included in the image data. Further, the waveform data acquisition unit 12 may specify a region associated to the chest portion 92 in the image by using, for example, a learned model that has learned by machine learning. The learned model is learned in such a way that the image of the subject is input and the region of the chest portion in the image is output. Further, the waveform data acquisition unit 12 may specify a region associated to the chest portion 92 by, for example, an operation by a user such as an instructor. In this case, the user may select a region associated to the chest portion 92 on the image of the subject 90 displayed on a touch panel by, for example, an operation such as tracing a region associated to the chest portion 92 with a finger on the touch panel. The waveform data acquisition unit 12 may also specify a region associated to the abdomen portion 94 in substantially the same manner as described above. Note that, for example, the waveform data acquisition unit 12 may specify a region associated to a sternum of the subject 90 (and a periphery thereof) as a chest region. Further, the waveform data acquisition unit 12 may specify a region associated to an umbilicus of the subject 90 (and a periphery thereof) as an abdominal region.

The photographed image 20g can also be displayed on the display device 30 under the control of the display control unit 16. In this case, for example, in the photographed image 20g, the subject image 90Im may be displayed in red, the chest region 92Im may be displayed in green, and the abdominal region 94Im may be displayed in blue, in such a way as to distinguish at least the chest region 92Im and the abdominal region 94Im from other regions.

The waveform data acquisition unit 12 has a function of specifying, as displacement amounts of the chest portion 92 and the abdomen portion 94, a displacement amount in at least one direction in the front-rear direction (vertical direction in the supine position), the left-right direction, and the up-down direction (direction from the top of the head toward the foot) of the subject 90. Therefore, the waveform data acquisition unit 12 has a function of specifying at least one direction of the front-rear direction, the left-right direction, and the up-down direction, and a function of specifying a displacement amount from a reference position in a target direction.

For example, the waveform data acquisition unit 12 may specify the front-rear direction and the left-right direction of the subject 90 by using the skeleton data. Alternatively, the waveform data acquisition unit 12 may recognize a head and lower limbs of the subject 90 and specify the up-down direction of the subject 90 from a direction of the center line of the subject 90 (an arrow A1 in FIG. 5) recognized from the recognized head and lower limbs. Further, the waveform data acquisition unit 12 may recognize both shoulders of the subject 90 and specify the left-right direction of the subject 90 from a direction of a line connecting the recognized both shoulders (an arrow A2 in FIG. 5). Then, the waveform data acquisition unit 12 may specify a direction perpendicular to the specified up-down direction and left-right direction as the front-rear direction. Further, the waveform data acquisition unit 12 may recognize a face of the subject 90 and specify a direction of the recognized face as the forward direction. Alternatively, when the subject 90 is in a state of supine position on a horizontal plane, the waveform data acquisition unit 12 may specify a direction along the vertical direction as the front-rear direction. In a case where the image data includes three-dimensional information, the front-rear direction can be acquired as a direction indicated by the distance information, i.e., the depth information.

In order to acquire a displacement amount in a certain direction, the waveform data acquisition unit 12 first specifies a reference position in the direction. In short, the waveform data acquisition unit 12 specifies each of the reference positions of the chest portion 92 and the abdomen portion 94 by using the photographed image 20g acquired when the subject 90 is in the rest state. Specifically, the waveform data acquisition unit 12 specifies the reference position of the chest portion 92 by using position information associated to the chest region specified in the photographed image 20g in the rest state. Similarly, the waveform data acquisition unit 12 specifies the reference position of the abdomen portion 94 by using position information associated to the abdominal region specified in the photographed image 20g in the rest state. Note that the waveform data acquisition unit 12 may specify the reference position of the torso (trunk) including the chest portion 92 and the abdomen portion 94 of the subject 90.

According to explanation on the front-rear direction, the waveform data acquisition unit 12 specifies reference positions of the chest portion 92 and the abdomen portion 94 in the front-rear direction. Herein, the waveform data acquisition unit 12 specifies a chest reference position, which is the reference position of the chest portion 92 in the front-rear direction. Similarly, the waveform data acquisition unit 12 specifies an abdominal reference position, which is the reference position of the abdomen portion 94 in the front-rear direction. When three-dimensional information is included in the image data, the chest reference position, the abdominal reference position, and the torso reference position can be acquired as depth information of a surface of the chest portion 92 at rest, and when the three-dimensional information is implicitly included, equivalent depth information can be acquired by analyzing the image data.

For example, the chest reference position may be an average position in the front-rear direction of the surface (front surface) of the chest portion 92 between exhalation and inspiration in the rest state. Similarly, the abdominal reference position may be an average position in the front-rear direction of a surface (front surface) of the abdomen portion 94 between exhalation and inspiration in the rest state. Note that the waveform data acquisition unit 12 may specify a reference position (body reference position) in the front-rear direction of the torso including the chest portion 92 and the abdomen portion 94.

Herein, the chest reference position may be, for example, an average position (first chest reference position) of a position in the front-rear direction (associated to “height” in the case of the supine position) at each position on the entire surface of the chest portion 92 at rest. In this case, the chest reference position may be indicated by one value. Alternatively, the chest reference position may be, for example, a position (second chest reference position) in the front-rear direction of one or more specific locations on the surface of the chest portion 92 at rest (e.g., a central location of the sternum of the chest portion 92). In this case, the chest reference position may be indicated by a number (N) of values associated to the number of specific locations.

Alternatively, the chest reference position may be, for example, a position in the front-rear direction (third chest reference position) of each of M locations arranged at predetermined intervals in the up-down direction (associated to the arrow A1 in FIG. 5) on the surface of the chest portion 92 at rest. In this case, the chest reference position may be indicated by M values. In the third chest reference position, “locations in the front-rear direction of the M locations arranged at predetermined intervals in the up-down direction” may be the average position (or the position on the foremost side) in the front-rear direction of the divided areas by dividing the chest region at predetermined intervals in the up-down direction. Alternatively, the chest reference position may be a position in the front-rear direction (fourth chest reference position) of each of n locations on the entire surface of the chest portion 92 at rest.

As for these matters that have been explained for the chest reference position, the same applies to the abdominal reference position and the torso reference position.

In explanation of the left-right direction, the waveform data acquisition unit 12 specifies the reference position (reference width) of each of the chest portion 92 and the abdomen portion 94 in the left-right direction (width direction) by using the photographed image 20g acquired when the subject 90 is in the rest state. The waveform data acquisition unit 12 specifies a chest reference width which is a reference width of the chest portion 92 in the left-right direction. Similarly, the waveform data acquisition unit 12 specifies an abdominal reference width which is a reference position of the abdomen portion 94 in the left-right direction. For example, the chest reference width may be an average width of the chest portion 92 between exhalation and inspiration in the rest state. Similarly, the abdominal reference width may be an average width of the abdomen portion 94 between exhalation and inspiration in the rest state. Herein, the chest reference width may be, for example, a distance between a left end and a right end of the chest portion 92 in a rest state. Similarly, the abdominal reference width may be, for example, a distance between a left end and a right end of the abdomen portion 94 in a rest state.

Note that the waveform data acquisition unit 12 may set a threshold Th1 of the width of the chest portion 92 at the time of exhalation. The threshold Th1 is associated to the width of the chest portion 92 when the breath is sufficiently exhaled during exhalation. Therefore, when the width of the chest portion 92 is narrowed to the threshold Th1 during exhalation, it can be said that internal rotation of the rib is sufficiently performed. Therefore, it can be said that the threshold Th1 is a target value of the chest width at the time of exhalation. The threshold Th1 is appropriately set by the instructor who has confirmed the breathing state of the subject 90. The threshold Th1 is a value smaller than the chest reference width. Therefore, when the chest reference width is 0, the threshold Th1 is a negative value.

Processing after acquiring the necessary reference position in this way will be explained. First, the imaging device 20 acquires an image of the subject 90 who is performing breathing training, and transmits the acquired image to the waveform data acquisition unit 12. Specifically, the instructor instructs the subject 90 to perform breathing training, and the imaging device 20 photographs the subject 90 in the state and transmits the photographed image to the waveform data acquisition unit 12. As a result, the waveform data acquisition unit 12 acquires the image data of the subject 90 who is performing the breathing training. The imaging device 20 acquires moving image data of the subject 90 who is performing breathing training or still image data of a predetermined time interval, and transmits the acquired data to the waveform data acquisition unit 12. Accordingly, the information processing apparatus 10 can detect a transition of each of the displacement amounts of the chest portion 92 and the abdomen portion 94.

The waveform data acquisition unit 12 can detect the displacement amount indicating a displacement of the target direction in the direction with respect to the reference position in the direction as follows. However, the method of detecting the displacement amount is not limited to the following method. The following displacement amount detection method may be executed for each frame of the acquired moving image data or for each of the acquired still image data. As a result, the waveform data acquisition unit 12 inputs the image data from the imaging device 20 at predetermined time intervals, analyzes the image data, detects each of the displacement amounts of the chest portion 92 and the abdomen portion 94 of the subject 90, and acquires time-series chest waveform data and abdominal waveform data indicating the respective changes.

The waveform data acquisition unit 12 detects the displacement amount from each of the reference positions of the chest portion 92 and the abdomen portion 94. Specifically, the waveform data acquisition unit 12 detects, for example, a change in the target direction in the chest portion 92 and the abdomen portion 94.

Hereinafter, a case in which the target direction is the front-rear direction will be explained as an example, but the same concept can be applied to other directions as well. When the target direction is the front-rear direction, the waveform data acquisition unit 12 detects a change in the position in the front-rear direction (associated to “height” in the case of the supine position). In the method of specifying the position after the change, as in the case of specifying the reference position, the chest region 92Im and the abdominal region 94Im may be specified from the image data, the target direction may be specified, and positions of the chest portion 92 and a surface (front surface) of the abdomen portion may be specified, and details thereof are omitted.

The waveform data acquisition unit 12 calculates an amount of change from the chest reference position with respect to the chest position which is the surface position of the chest portion 92 and is specified as described above, and an amount of change from the abdominal reference position with respect to the abdominal position which is the surface position of the abdomen portion and is specified as described above.

Herein, when the chest position is on a front side of the chest reference position, i.e., when the chest portion 92 is expanded more than at rest, a sign of the displacement amount is positive (+). On the other hand, when the chest position is located behind the chest reference position, i.e., when the chest portion 92 is contracted more than at rest, the sign of the displacement amount is negative (-). Therefore, the waveform data acquisition unit 12 calculates the displacement amount of the chest portion 92 by subtracting the value of the chest reference position from the value of the chest position.

Further, when the abdominal position is on a front side of the abdominal reference position, i.e., when the abdomen portion 94 is expanded more than at rest, the sign of the displacement amount becomes positive (+). On the other hand, when the abdominal position is located rearward of the abdominal reference position, i.e., when the abdomen portion 94 is contracted more than at rest, the sign of the displacement amount is negative (-). Therefore, the waveform data acquisition unit 12 calculates the displacement amount of the abdomen portion 94 by subtracting the value of the abdominal reference position from the value of the abdominal position.

In this manner, the waveform data acquisition unit 12 detects the displacement amount from each of the reference positions of the chest portion 92 and the abdomen portion 94. The waveform data acquisition unit 12 executes the detection of the displacement amount at predetermined time intervals, and thereby, it is possible to acquire, for example, time-series chest waveform data and abdominal waveform data indicating a change in the displacement amount of the chest portion 92 and the abdomen portion 94 as illustrated in FIG. 6. In the example of FIG. 6, the amount of change in the chest portion 92 and the amount of change in the abdomen portion 94 from the start time of the breathing training are illustrated. In FIG. 6, zero of the amount of change represents each of the chest reference position and the abdomen reference position. The waveform data acquisition unit 12 passes the acquired chest waveform data and abdominal waveform data to the division processing unit 13.

An example of processing of acquiring time-series chest waveform data and abdominal waveform data from image data captured by the imaging device 20 has been described above with reference to FIGS. 5 and 6. The chest waveform data and the abdominal waveform data are not limited to such an acquisition method. For example, the imaging device 20 may be a two-dimensional camera (e.g., an RGB camera, etc.) and may be arranged to photograph from a lateral direction of the subject 90. In this case, the waveform data acquisition unit 12 can acquire the chest waveform data and the abdominal waveform data by analyzing the image data, calculating the vertical movement of the chest portion 92 and the abdomen portion 94 from the change between the images, and arranging calculation results in time series.

Further, the display system 100 is not limited to being provided with the imaging device 20 functioning as a distance image sensor or the like. The display system 100 may include other types of sensors capable of acquiring the abdominal waveform data and the chest waveform data, or capable of acquiring the abdominal waveform data and the chest waveform data by analysis in the information processing apparatus 10.

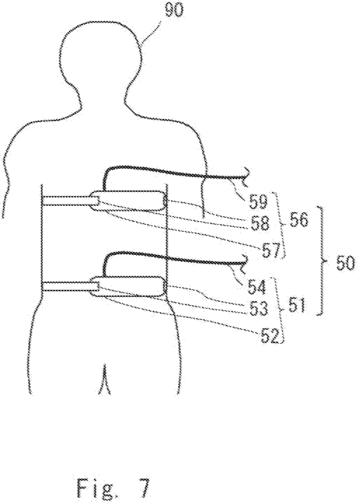

Examples of such a sensor include a band-type respiration measurement sensor (waveform measurement sensor) acquired by measuring the chest waveform data and the abdominal waveform data itself. With reference to FIG. 7, an example of processing of acquiring time-series chest waveform data and abdominal waveform data in a case where a band-type waveform acquisition sensor is used instead of the imaging device 20 will be explained. FIG. 7 is a top view schematically illustrating an example of a band-type waveform acquisition sensor that can be adopted in place of the imaging device 20 in the display systems of FIGS. 3 and 4.

As illustrated in FIG. 7, the band-type waveform acquisition sensor 50 may include an abdominal waveform acquisition sensor 51 and a chest waveform acquisition sensor 56. The abdominal waveform acquisition sensor 51 may include a sensor portion 52, a band 53, and a connection cable 54. The sensor portion 52 is attached to the abdomen portion of the subject 90 by the band 53. The chest waveform acquisition sensor 56 may include a sensor portion 57, a band 58, and a connection cable 59. The sensor portion 57 is attached to the chest portion of the subject 90 by the band 58. Note that the cables 54 and 59 can be combined into one piece, and the cables 54 and 59 can be eliminated by providing the waveform acquisition sensor 50 with a wireless communication circuit.

In this case, the waveform data acquisition unit 12 of the information processing apparatus 10 acquires the chest waveform data and the abdominal waveform data measured by the band-type respiration measurement sensor, and passes the data to the division processing unit 13. The chest waveform data and the abdominal waveform data acquired herein may also be data as illustrated in FIG. 6. Note that the band-type respiration measurement sensor is not limited to the one having the configuration and shape illustrated in FIG. 7, and a respiration measurement sensor capable of measuring a breathing waveform other than the band-type sensor may be employed.

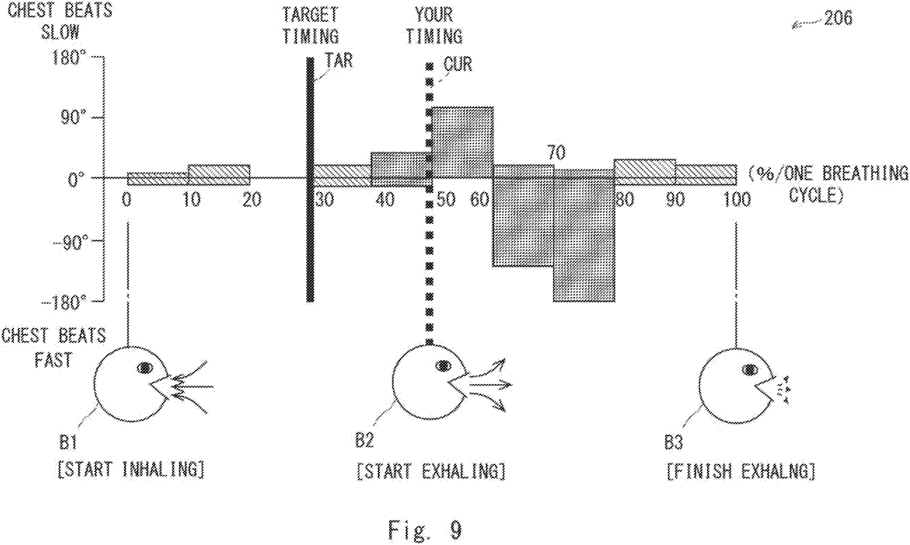

Next, an example of processing in the display control unit 16 will be explained with reference to FIGS. 8 and 9. FIG. 8 is a diagram illustrating an example of a distribution of phase differences displayed on the display device 30 under the control of the information processing apparatus 10. FIG. 9 is a diagram illustrating another example of the distribution of the phase differences displayed on the display device 30 under the control of the information processing device 10. FIGS. 8 and 9 are examples each illustrating a distribution of results using abdominal waveform data and chest waveform data different from each other, and FIG. 8 illustrates a distribution based on the latest data of the same subject 90, and FIG. 9 illustrates a distribution based on past data.

First, in the phase difference calculation unit 14, as described above, the abdominal waveform data and the chest waveform data in each phase are Hilbert transformed and calculate an instantaneous phase, normalized in such a way as to express one breathing cycle at 100%, and averaged in units of 10%. The display control unit 16 controls the display device 30 to display the distribution of the average phase difference in units of 10% calculated by the phase difference calculation unit 14 in this manner. Herein, the display control unit 16 also performs processing of graphing the above distribution as illustrated in FIG. 8, i.e., processing of generating a graph representing an average phase difference in a section of a 10% interval. Thus, a graph 204 of the distribution illustrated in FIG. 8 can be displayed on the display device 30.

Further, as illustrated in FIG. 8, the graph 204 may include information indicating a timing CUR of the switching between the expiratory phase and the inspiratory phase by the subject 90 and a timing TAR of the switching between the target expiratory phase and inspiratory phase. Any of the switching timings may refer to timings at which exhalation is started. In addition, although an example in which the timing CUR is displayed by a line and it is explicitly stated that it is “your timing” is given, a display mode of the timing CUR is not limited to this. In addition, although an example in which the timing TAR is displayed by a line and it is explicitly stated as “target timing” is given, a display mode of the timing TAR is not limited to this.

Further, as described above, since the timing CUR indicates the timing at which the subject 90 has started exhalation, it can be added an illustration B2 that makes this easy to understand to the graph as illustrated in FIG. 8. Similarly, an illustration B1 indicating a start of inhaling and an illustration B3 indicating an end of exhaling may be added to the graph. The illustrations B1 to B3 are not limited to the example illustrated in FIG. 8, for example, illustrations illustrating timings in a degree of bulging of the balloon.

In addition, the timing TAR may be automatically set to a timing such that a current state is determined based on past data or the like of the subject 90 and matches the current state as a target, or may be manually set by the instructor or the subject 90. As described above, the information processing apparatus 10 can display the start timing of the target expiratory phase on the display device 30 under the control of the display control unit 16, and can also include a setting function (setting unit) of setting the target.

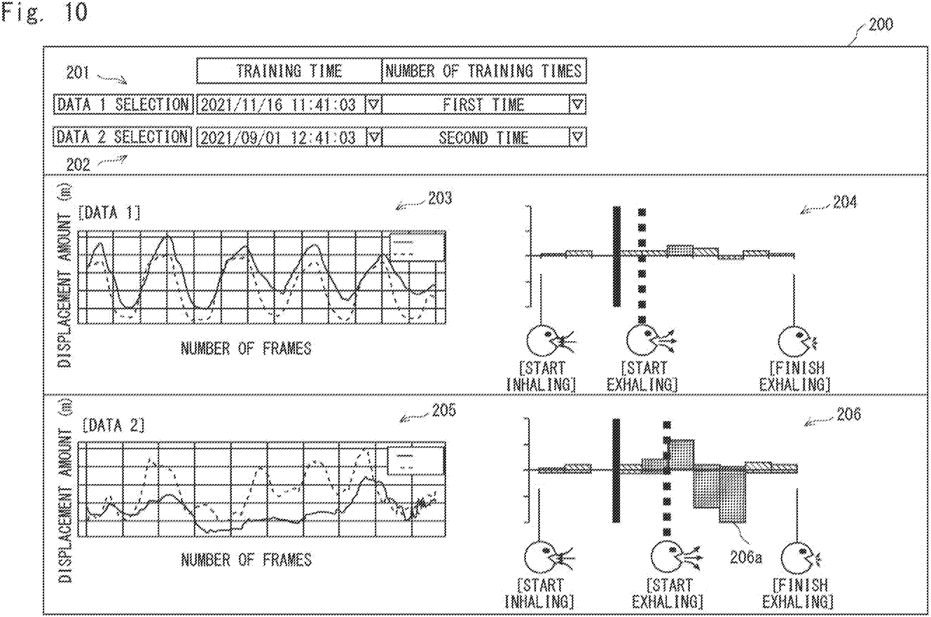

As in the example of FIG. 8, the display control unit 16 can control the display device 30 to display a graph 206 indicating the distribution of the phase difference based on the past data of the subject 90, as illustrated in FIG. 9. Further, the display control unit 16 can also perform control in such a way that the graph 204 in FIG. 8 and the graph 206 in FIG. 9 are displayed side by side on the display device 30. This will be described later with reference to FIG. 10.

Further, as illustrated in FIGS. 8 and 9, the display control unit 16 may generate the graphs 204 and 206 in such a way that a portion having a large phase difference for each section is displayed in a display mode different from a portion having a small phase difference. Note that in the example of FIG. 8, a pattern (hatching) is different from another section in one section of 50% to 60%, and in the example of FIG. 9, a pattern is different from other sections in four sections of 40% to 80%. However, it is also possible to generate a graph in such a way that the display modes are the same in all sections.

Herein, “display modes are the same” may be, for example, the same type of pattern (hatching), but is not limited thereto. In short, the “display mode” is not limited to “pattern”. For example, the display mode may be a color representation, a gray scale (shade of black), or the like. In these cases, the display control unit 16 may generate a graph such that a portion having a large phase difference in each section is displayed in a color representation or gray scale different from a small portion. For example, in the graphs 204 and 206, a section in which the phase difference is determined to be large may be displayed in red, and other sections may be displayed in blue. Alternatively, in the graphs 204 and 206, a section in which the phase difference is determined to be large can be displayed in a dark manner, and other sections can be displayed in a light manner.

In a case of adopting a different display mode as described above, the phase difference calculation unit 14 or the display control unit 16 may determine presence or absence of a phase difference in each section, determine whether or not the phase difference is equal to or larger than a predetermined threshold in the case where there is a phase difference, and may select the display mode by using the result. Note that it is only necessary to determine whether or not the phase difference is equal to or greater than a predetermined threshold value without determining the presence or absence of the phase difference.

An example of the threshold processing will be explained. When L is a positive integer and L breaths are included in one series of the abdominal waveform data and the chest waveform data to be processed, L pieces of phase difference data exist in each section. The phase difference calculation unit 14 or the display control unit 16 determines that the phase difference is large when the number of times exceeding the threshold Th of the phase difference is P times or more for each section, and determines that the phase difference is small when the number of times exceeding the threshold Th is less than P times.

Although an example in which one predetermined threshold value is used has been given, a plurality of predetermined threshold values can be set, and thus a change in the display mode in three or more stages can be represented in a graph. As described above, the display control unit 16 can perform the control of displaying in a different display mode according to a magnitude of the phase difference.

With the display control as described above, in the display device 30, the value of the phase difference can be displayed for each section of the breathing cycle of the subject 90. The breathing cycle includes the expiratory phase and the inspiratory phase of the subject 90, and in the graphs 204 and 206 of FIGS. 8 and 9, the phase difference is displayed for a plurality of sections in both the expiratory phase and the inspiratory phase. As described above, by displaying a plurality of phase difference values for both phases, by increasing the number of phase differences to be further displayed, it is possible to feedback information indicating at which timing the synchronization is not achieved to the subject 90 directly or via the instructor in detail. In short, by presenting such detailed information, it is possible to promote understanding of synchrony during exhalation and inspiration by more finely providing feedback to the subject 90 directly or through the instructor.

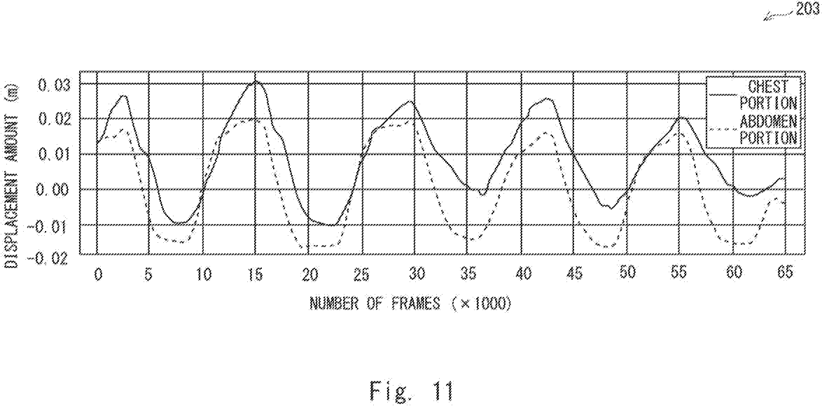

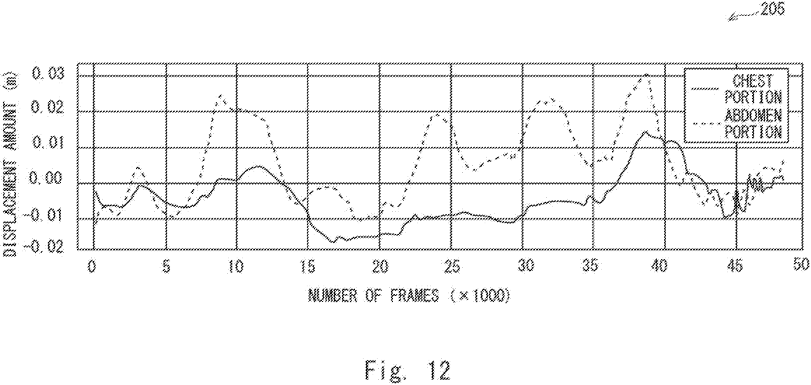

Next, a display example of the distribution of the phase difference will be explained with reference to FIGS. 10 to 12. FIG. 10 is a diagram illustrating an example of an image including the distribution of phase differences displayed on the display device 30 under the control of the information processing apparatus 10. FIG. 11 is a graph illustrating an example of the chest waveform data and the abdominal waveform data of data 1 in FIG. 10, and FIG. 12 is a graph illustrating an example of the chest waveform data and the abdominal waveform data of data 2 in FIG. 10.

In the example described herein, it is assumed that the display control unit 16 performs control of displaying for comparison, on the display device 30, the distribution of the phase difference calculated based on the past abdominal waveform data and the past chest waveform data of the subject 90.

The display control unit 16 can perform control of displaying a display image 200 illustrated in FIG. 10 on the display device 30. The display image 100 may include a selection region 201 for selecting data 1, which are first data, and a selection region 202 for selecting data 2, which are second data. Herein, an example will be given in which the selected data 1 are the abdominal waveform data and the chest waveform data used at the time of generating the graph 204 in FIG. 8, and the selected data 2 are the abdominal waveform data and the chest waveform data used at the time of generating the graph 206 in FIG. 9. In short, in this example, explanation is made on the assumption that the data 1 are automatically selected as the display target for the breathing training immediately after the subject 90 ends.

As described above, the information processing apparatus 10 may include a selection unit that selects the past abdominal waveform data and the past chest waveform data. Herein, the past abdominal waveform data and the past chest waveform data are data for comparison, but may include data immediately after the end of the breathing training. In other words, it is sufficient that two pieces of data can be selected for comparison. Further, the selection target is not limited to two data, and may be three or more data, and in this case, the graph or the like of the display target is also increased by that amount.

The selection unit may include a control unit 11 and a storage unit 15, and may select past data to be displayed by the control unit 11 from data group stored in the storage unit 15. Note that the data group may be stored in a server device connected to the information processing apparatus 10, i.e., the information processing apparatus 10 may distribute a part of functions of the storage unit 15 to the server device.

Further, although not illustrated, the selection unit may include an operation unit that accepts an operation from the subject 90 or the instructor, and the control unit 11 may select past data to be displayed from the data group in accordance with an operation accepted by the operation unit. For example, as the data 2, data about past breathing training of the subject 90 can be manually selected, and the selection regions 201 and 202 are included in the display image 200 in such a way that such a selection operation can be performed. In the selection regions 201 and 202, the training time and the number of times of training (information indicating the number of times) can be selected as a pull-down menu.

In addition, when the operation unit is not used, the control unit 11 can automatically select the past data for comparison that satisfy a predetermined condition from the data groups stored in the storage unit 15. In other words, the selection unit can be configured to search for and select past abdominal waveform data and chest waveform data that satisfy a predetermined condition. For example, as the data 2, data for past breathing training of the subject 90 may be automatically selected.

As the predetermined condition, for example, a day closest to the relevant day one month ago or a day closest to the relevant day one week ago can be adopted. Alternatively, as a predetermined condition, it may be adopted that it is the closest past day from the current breathing training. Alternatively, as the predetermined condition, it is also possible to adopt that the phase difference is the largest data in the past.

The display image 200 includes a graph 203 of the abdominal waveform data and the chest waveform data and a graph 204 in FIG. 8, for the data 1 to be displayed. In FIG. 10, values of the graph 203 and the like are omitted for convenience, but the graph 203 is illustrated in FIG. 11. The display image 200 includes a graph 205 of the abdominal waveform data and the chest waveform data and a graph 206 of FIG. 9, for the data 2 to be displayed. In FIG. 10, values of the graph 205 and the like are omitted for convenience, but the graph 205 is illustrated in FIG. 12.

In this way, the result of the graph or the like based on the data for comparison and the result of the graph or the like based on the data for the breathing training completed this time are displayed, whereby the subject 90 can know a result of this breathing training at a glance directly or through the instructor.

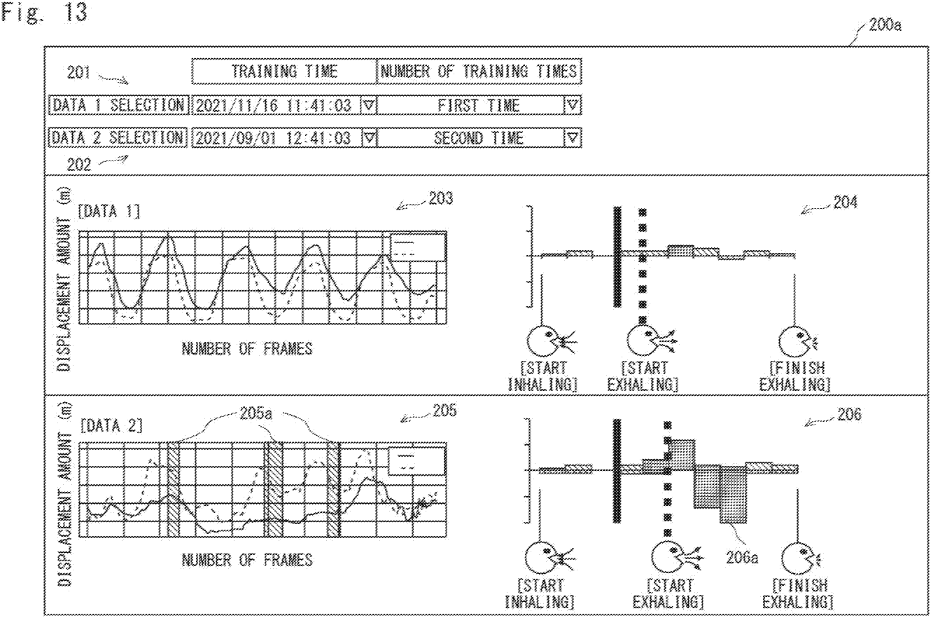

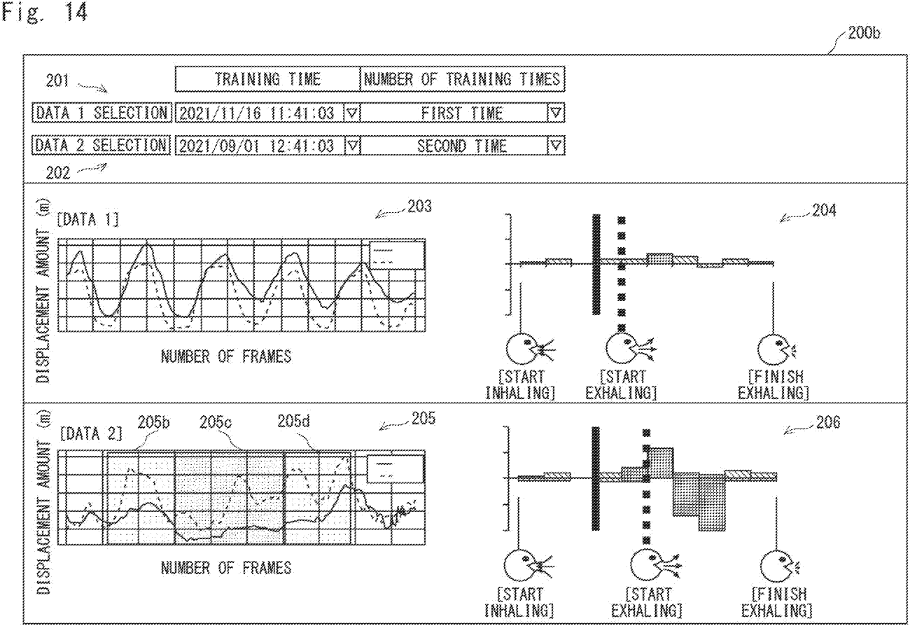

Next, a display example different from that of FIG. 10 will be explained with reference to FIGS. 13 and 14. Both of FIGS. 13 and 14 are examples of images including a distribution of phase differences displayed on the display device 30 under the control of the information processing apparatus 10, and are diagrams illustrating different examples from that of FIG. 10.

A display image 200a illustrated in FIG. 13 is an example of an image displayed when an operation of selecting an optional section of the graph 204 and the graph 206 is made receivable in the display image 200 in FIG. 10 and a section 206a is selected.

In the display image 200a in FIG. 13, a region in which the phase difference in the graph 206 is larger than a predetermined threshold value is drawn in such a way as to be displayed as a highlight region 205a in the section 206a illustrated in FIG. 13. The phase difference calculation unit 14 or the display control unit 16 can execute the determination with the predetermined threshold value as described above.

The highlight region 205a is not limited to the highlight display as long as it has a display mode that can be distinguished from other regions. As illustrated in FIG. 13, the highlight region 205a may be a region in which a region larger than a predetermined threshold value is highlighted in a single color regardless of the degree of phase difference, but it is not limited thereto. For example, the highlight region 205a may perform determination at a plurality of predetermined thresholds, and may be displayed in gradations according to the degree of the phase difference.

Of course, when a section different from the section 206a is selected in the graph 206, an associated portion different from the position indicated by the highlight region 205a in the graph 205 can be displayed in a single color or a gradient. When a certain section is selected in the graph 204, the associated portion may be displayed in a single color or a gradient in the graph 203.

A display image 200b illustrated in FIG. 14 is a display image 200 illustrated in FIG. 10 in which a region in which the phase difference is larger than a predetermined threshold value is displayed in a gradation in which a dark color is set in accordance with the magnitude of the phase difference. Herein, for convenience, an example is given in which two gradation displays are performed, but three or more gradation displays can be performed, and gradation displays can also be performed only by prepared gradation levels. In this case, the gray level to be displayed may be determined in advance according to the magnitude of the phase difference.

In the example of FIG. 14, the graph 205 results in including the gradation regions 205b, 205c, and 205d for the data 2 that are the past data, whereas the graph 203 for the data 1 that are the current data does not include the gradation region. As described above, by performing a display in which a portion having a large phase difference is particularly close-up by comparison with past data, the subject 90 can actually feel a result through the present breathing training directly or through the instructor.

In addition, in the display image 200b, it is also possible to switch between the gradation display and the non-display by pressing a selection button (not illustrated) or the like.

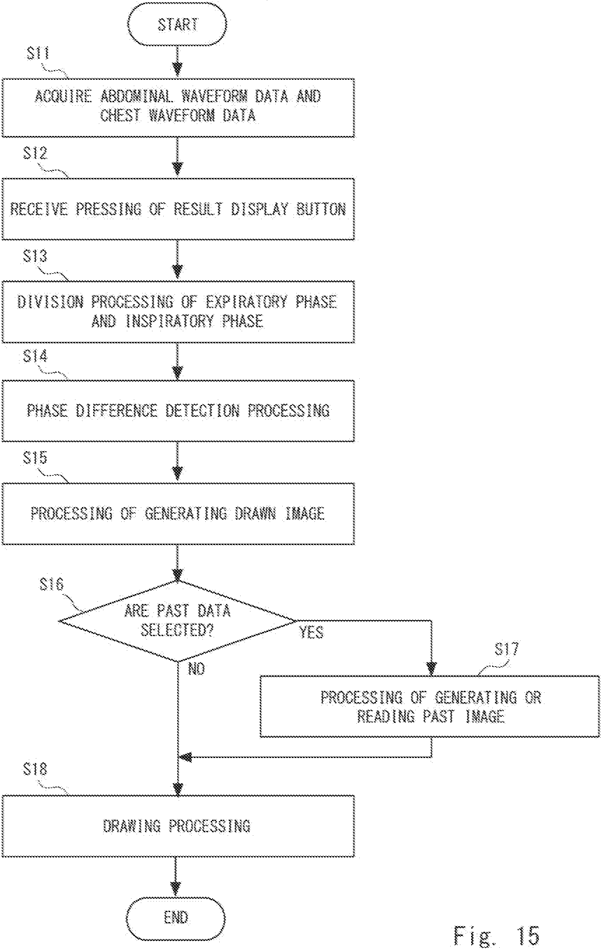

Next, an example of processing in a display system 100 will be described with reference to FIG. 15. FIG. 15 is a flowchart for explaining an example of processing in the display system 100. However, the processing in the display system 100 is not limited to the example explained herein.

First, the information processing apparatus 10 acquires the abdominal waveform data and the chest waveform data for the subject 90 who performs the breathing training (step S11). Next, the information processing apparatus 10 receives pressing of a result display button that is not illustrated (step S12). This reception can be performed by the display device 30, and the information can be passed to the information processing apparatus 10.

Next, the information processing apparatus 10 divides the data acquired in step S11 into an expiratory phase and an inspiratory phase (step S13). Next, the information processing apparatus 10 calculates (detects) a phase difference between the abdominal waveform data and the chest waveform data in each of the expiratory phase and the inspiratory phase from the divided data (step S14). Steps S13 and S14 may be executed before step S12.

Next, the information processing apparatus 10 generates a drawn image such as the graph 204 of FIG. 8, based on the detected phase difference (step S15). The information processing apparatus 10 determines whether or not past data for comparison have been selected (i.e., whether or not data for comparison are displayed) (step S16).

In the case of YES in step S16, the information processing apparatus 10 performs processing of generating or reading a past image (step S17), adds to the drawing image generated in step S15, and performs drawing for display (step S18). In step S18, the image drawn by the information processing apparatus 10, for example, the display image 200 in FIG. 10 or the display image 200b in FIG. 14 is further displayed on the display device 30, and the processing ends. In the case of NO in step S16, the information processing apparatus 10 proceeds to step S18 without passing through step S17, performs drawing for display by using the drawn image generated in step S15, causes the display device 30 to display the drawn image, and ends the processing.

As described above, according to the present example embodiment, it is possible to cause the subject 90 to visually recognize information indicating at which timing the synchronization between the abdomen portion and the chest portion of the subject performing the breathing training is not achieved, either directly or via the instructor. In addition, this enables timely feedback of this information to the subject or an instructor who sends advice to the subject, thereby enabling more effective breathing training to be performed.

Further, according to the present example embodiment, as explained as an effect of the first example embodiment, it is possible to provide effective guidance by being used by an instructor such as a therapist at the time of rehabilitation in a medical institution or breathing exercise practice in a healthcare service. Further, by implementing the information processing apparatus 10 or the information processing apparatus 10 and the display device 30 as a terminal device to be used by the subject, the subject can receive remote instruction from the instructor and perform voluntary training while being at home. In particular, by mounting the function of the information processing apparatus 10 as an application or the like in a portable terminal device such as a tablet terminal to be used by a subject, it becomes easier for the subject 90 to perform breathing training. The imaging device 20 can also use a camera or the like mounted on the terminal device.

Further, in the present example embodiment, it has been mainly explained that the abdominal waveform data and the chest waveform data are data indicating the displacement amount in the front-rear direction, but the same processing can be performed on the data indicating the displacement amount in the left-right direction or the data indicating the displacement amount in the up-down direction. However, it can be appropriately changed the threshold value or the like to be used for the determination with respect to the phase difference in accordance with the data to be used.

Further, in the present example embodiment, the supine position has been explained as an example of the posture at the time of photographing the image data and acquiring the data, i.e., the posture of the breathing training, but it can also be performed, for example, in a sitting position, a standing position, a knee standing position, a supine position, and a leg raising position according to the exercise purpose. However, the installation location of the imaging device 20, the threshold to be used for the determination with respect to the phase difference, and the like may be changed as appropriate in accordance with the posture.

Further, the display system 100 is provided with a sensor for detecting a proportion of carbon dioxide in the nose or the like of the subject 90, and the expiratory phase and the inspiratory phase can be easily divided by configuring the display system in such a way as to perform the expiratory analysis, based on the detection result of the sensor. In this case, it is not necessary to provide the division processing unit 13. However, even when such a sensor is not prepared, it is possible to separate the expiratory phase and the inspiratory phase from the average waveform in the division processing unit 13 as described above, providing the division processing unit 13, simplification of the system configuration and a time and effort of mounting the sensor can be omitted, it can be said to be advantageous.

### Third Example Embodiment

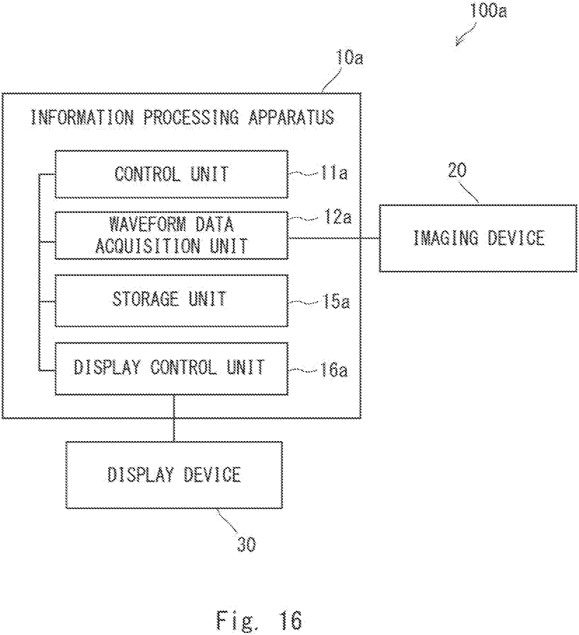

Although a third example embodiment will be mainly explained with reference to FIGS. 16 to 19, various examples explained in the first and second example embodiments can be applied. First, a configuration example of an information display system (hereinafter, simply referred to as a display system) including an information processing apparatus according to the present example embodiment will be explained with reference to FIG. 16. FIG. 16 is a block diagram illustrating a configuration example of a display system including the information processing apparatus according to the third example embodiment. An example of appearance of a display system 100a illustrated in FIG. 16 is the same as that illustrated in FIG. 4.

As illustrated in FIG. 16, the display system 100a according to the present example embodiment includes an information processing apparatus 10a that is an example of the information processing apparatus 1 illustrated in FIG. 1, at least one imaging device 20, and at least one display device 30. The information processing apparatus 10a is communicably connected to the imaging device 20 and the display device 30 via a wired or wireless network.

As illustrated in FIG. 16, the information processing apparatus 10a may include a control unit 11a, a waveform data acquisition unit 12a, a storage unit 15a, and a display control unit 16a. The control unit 11a, the waveform data acquisition unit 12a, and the storage unit 15a are associated to the control unit 11, the waveform data acquisition unit 12, and the storage unit 15 in FIG. 3, respectively. However, the processed data to be stored in the storage unit 15a may be different from the processed data to be stored in the storage unit 15.

Similarly to the waveform data acquisition unit 12, the waveform data acquisition unit 12a inputs image data from the imaging device 20 at predetermined time intervals, for example, analyzes the image data, and detects each of the displacement amounts of the chest portion 92 and the abdomen portion 94 of the subject 90. Also in the present example embodiment, the waveform data acquisition unit 12a can acquire not only the waveform data from the image data but also the abdominal waveform data and the chest waveform data from other types of sensors such as the band-type waveform acquisition sensor 50 in FIG. 7, for example.

The waveform data acquisition unit 12a can acquire time-series chest waveform data and abdominal waveform data indicating each change as the displacement amounts of the chest portion 92 and the abdomen portion 94, and the acquired chest waveform data and the abdominal waveform data are passed to the display control unit 16a or passed to the storage unit 15a. The chest waveform data and the abdominal waveform data passed to the storage unit 15a are read out by the display control unit 16a at the time of display control.

In the present example embodiment, the display control unit 16a performs control of displaying, on the display device 30, the time-series displacement value of the abdomen portion 94 indicated by the abdominal waveform data and the time-series displacement value of the chest portion 92 indicated by the chest waveform data by plotting one as a vertical axis and another as a horizontal axis. The time-series displacement value of the abdomen portion 94 is series information of the abdominal position, and the time-series displacement value of the chest portion 92 is series information of the chest position.

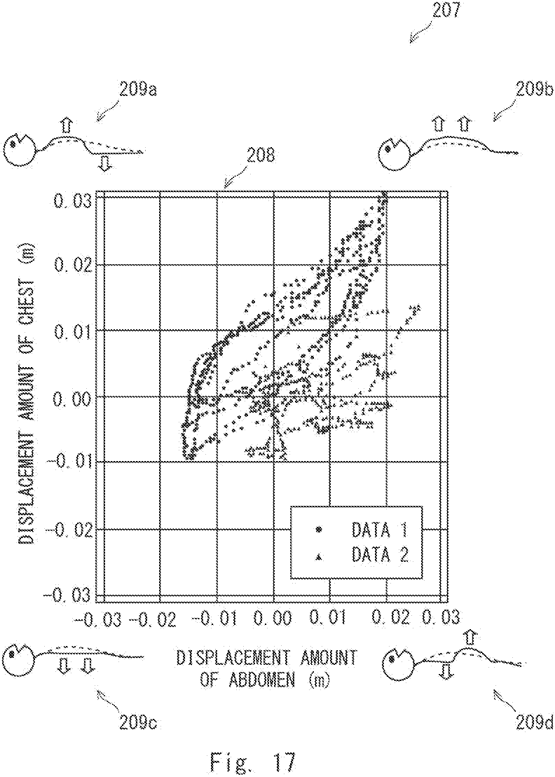

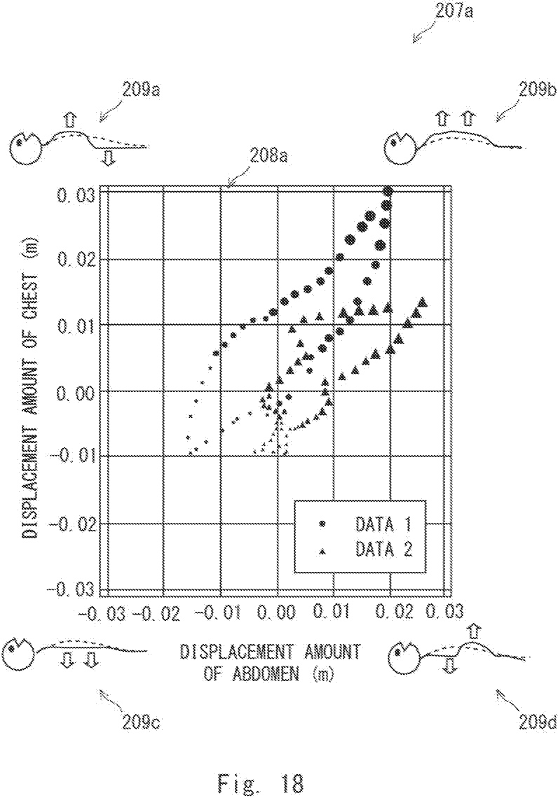

An example of such a display will be explained with reference to FIGS. 17 and 18. FIG. 17 is a diagram illustrating an example of transition of displacement to be displayed on the display device 30 under the control of the information processing apparatus 10a in the display system 100a in FIG. 16. FIG. 18 is a diagram illustrating another example of the transition of the displacement to be displayed on the display device 30 under the control of the information processing apparatus 10a in the display system 100a in FIG. 16.

The display image 207 illustrated in FIG. 17 is an example of an image to be displayed on the display device 30 by the display control unit 16a, and includes a graph 208 in which one of a time-series displacement value of the abdomen portion 94 and a time-series displacement value of the chest portion 92 is plotted as a vertical axis and another thereof is plotted as a horizontal axis. The graph 208 is a graph plotting the change of each displacement value, and in this example, the displacement amount of the chest portion 92 is plotted as the vertical axis and the displacement amount of the abdomen portion 94 is plotted as the horizontal axis.

Although the graph 208 illustrates the positions of the data 1 illustrated in FIG. 11 with a round marker and the data 2 illustrated in FIG. 12 with a triangular marker, the selected region of the data can also be simultaneously displayed as illustrated in FIG. 10. In addition, the display mode of the marker is not limited to a circle or a triangle, and it is sufficient that data to be displayed (two in this example) can be distinguished.

In the graph 208, it can be seen that the plot position for the data 1 resembles a steadily increasing plot pattern, which is a generally preferred breathing plot pattern, and that abdomen portion 94 and chest portion 92 can be generally well synchronized. On the other hand, in the graph 208, it can be seen that the plot position of the data 2, which are the past data, is far from such a steadily increasing plot pattern, and thus the abdomen portion 94 and the chest portion 92 cannot be well synchronized with each other. As described above, in the present example embodiment, the breathing waveform data of the chest portion 92 and the abdomen portion 94 are input, the position series information is plotted as the position vertical axis and horizontal axis of each waveform, and the synchronicity between the chest portion 92 and the abdomen portion 94 can be displayed on the display device 30.

Further, as illustrated in FIG. 17, the displayed images 207 may include illustrations 209a, 209b, 209c, and 209d at or near a vertex of the frame of the graph 208. Each of the illustrations 209a to 209d is an illustration illustrating a face of a person who is regarded as the subject 90, a dot row indicating a reference position of the torso (including a reference position of the chest portion and the abdomen portion), and a torso position (including a measurement position of the chest portion and the abdomen portion) associated to a state of each vertex. Each of the illustrations 209a to 209d also indicates arrows indicating directions of movements of the chest and abdomen.

For example, the illustration 209a indicates that the amount of change in the chest portion is large toward a + side and the amount of change in the abdomen portion is large toward a - side, and the illustration 209b indicates that both the amount of change in the chest portion and the amount of change in the abdomen portion are large toward the + side. In addition, the illustration 209c indicates that the amount of change in the chest portion and the amount of change in the abdomen portion are both large on the - side, and the illustration 209d indicates that the amount of change in the chest portion is large on the - side and the amount of change in the abdomen portion is large on the + side. By including the illustrations 209a to 209d in the associated locations of the display image 207, the subject 90 can check what state the subject is in by comparing with the plot.

Further, it is desirable to change the graph 208 in such a way that an elapse of time can be known, assuming that the synchronization is not successful as in the data 2. Therefore, the display control unit 16a can also perform control of displaying markers representing a time-series displacement value of the abdomen portion 94 and a time-series displacement value of the chest portion 92 in such a way as to have a different display mode between a value close to the peak of exhalation and a value close to the peak of inspiration. In this example, the display control unit 16a changes a size of the marker in such a way that the closer to the expiratory peak is larger and the closer to the inspiratory peak is smaller. However, for example, the display control unit 16a may change the color of the marker in such a way that the closer to the expiratory peak, the darker the color, and the closer to the inspiratory peak, the lighter the color.

Whether it is close to the expiratory peak or close to the inspiratory peak can be determined by using, for example, the method of dividing the expiratory phase and the inspiratory phase, which is explained in the second example embodiment, but the determination method is not limited thereto. For example, as a result of performing an exhalation analysis by providing a sensor for detecting a proportion of carbon dioxide in the nose or the like of the subject 90, it is possible to determine whether or not it is close to each peak.

With such a configuration, in the information processing apparatus 10a, the breathing waveform data of the chest portion 92 and the abdomen portion 94 are input, and position series information is plotted as the position vertical axis and the horizontal axis of each waveform, and not only the synchrony between the chest portion 92 and the abdomen portion 94 but also the magnitude of the movement can be displayed on the display device.

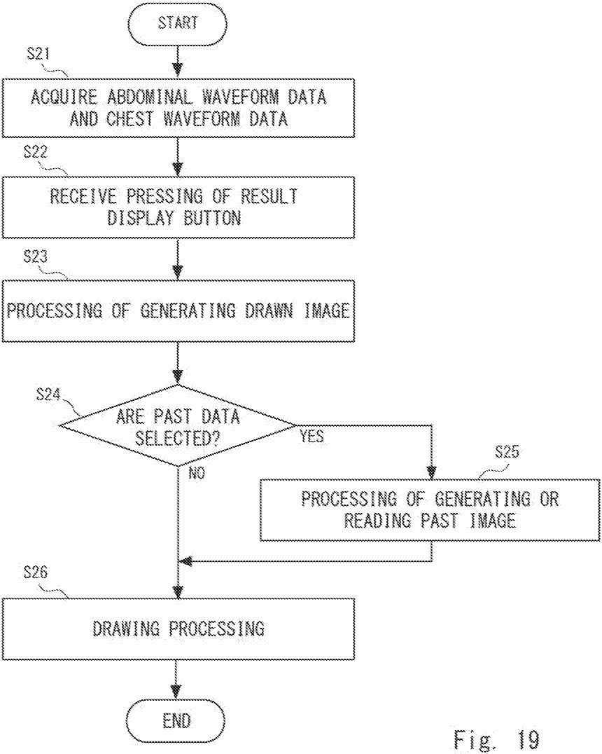

Next, an example of processing in the display system 100a will be explained with reference to FIG. 19. FIG. 19 is a flowchart for explaining an example of processing in the display system 100a. However, the processing in the display system 100a is not limited to the example explained herein.

First, similarly to steps S11 and S12 in FIG. 15, the information processing apparatus 10a acquires the abdominal waveform data and the chest waveform data for the subject 90 who performs the breathing training (step S21), and receives pressing of a result display button that is not illustrated (step S22). This reception can also be performed by the display device 30, and the information can be passed to the information processing apparatus 10a.

Next, the information processing apparatus 10a generates a drawn image such as the graph 208 of FIG. 17 for the data acquired in step S11 (step S23). The information processing apparatus 10a determines whether or not past data for comparison has been selected (i.e., whether or not data for comparison are displayed) (step S24).

In a case of YES in step S24, the information processing apparatus 10a performs processing of generating or reading a past image (step S25), adds to the drawing image generated in step S23, and performs drawing for display (step S26). In step S26, the image drawn by the information processing apparatus 10a, for example, the display image 207 in FIG. 17 or the display image 207a in FIG. 18 is further displayed on the display device 30, and the processing ends. In the case of NO in step S24, the information processing apparatus 10a proceeds to step S26 without passing through step S25, performs drawing for display by using the drawn image generated in step S23, causes the display device 30 to display the drawn image, and ends the processing.

As described above, according to the present example embodiment, it is possible to cause the subject 90 to visually recognize the information indicating at which timing the synchronization between the abdomen portion and the chest portion of the subject performing the breathing training is not achieved, as a variation in the plot position, or the like, directly or via the instructor. In addition, in order to clarify this information, an ideal line that is steadily increasing in the display image 207 or the display image 207a may be included together with the explanation. In addition, this enables timely feedback of this information to the subject or an instructor who sends advice to the subject, thereby enabling more effective breathing training to be performed.

Further, according to the present example embodiment, as explained as an effect of the first example embodiment, it is possible to provide effective guidance by being used by an instructor such as a therapist at the time of rehabilitation in a medical institution or breathing exercise practice in a healthcare service. Further, by implementing the information processing apparatus 10a or the information processing apparatus 10a and the display device 30 as a terminal device to be used by the subject, the subject can receive remote instruction from the instructor and perform voluntary training while being at home. In particular, by mounting the function of the information processing apparatus 10a as an application or the like in a portable terminal device such as a tablet terminal to be used by the subject, it becomes easier for the subject 90 to perform breathing training. The imaging device 20 can also use a camera or the like mounted on the terminal device.

Also in the present example embodiment, similarly to the second example embodiment, the explanation has been given on the assumption that the abdominal waveform data and the chest waveform data are mainly data indicating the displacement amount in the front-rear direction. However, also in the present example embodiment, the same processing can be performed on data indicating the displacement amount in the left-right direction or data indicating the displacement amount in the up-down direction.

Further, also in the present example embodiment, the supine position has been explained as an example of the posture at the time of photographing the image data and acquiring the data, i.e., the posture of the breathing training, but it can also be performed, for example, in the sitting position, the standing position, the knee standing position, the supine position, and the leg raising position according to the exercise purpose. However, an installation location of the imaging device 20, the illustrations 209a to 209d, and the like may be appropriately changed in accordance with the posture.

As explained above, the present example embodiment can be executed independently from the processing of calculating the phase difference between the abdominal waveform data and the chest waveform data in each of the expiratory phase and the inspiratory phase explained in the first and second example embodiments. In other words, the information processing apparatus 10a according to the present example embodiment enables not to include a function of calculating such a phase difference.

However, the information processing apparatus 10a according to the present example embodiment may be configured to include a function of calculating such a phase difference, i.e., may also have a function of the information processing apparatus 10. In this case, for example, the display image 207 in FIG. 17 or the display image 208 in FIG. 18 may be displayed simultaneously with the graph 204 of FIG. 8, the display images 200, 200a and 200b in FIGS. 10 to 13, and the like. Further, the information processing apparatus 10a can be configured to switch the display image, for example, from the display image 200 to the display image 207 by providing a function of switching the display image.

### Modification

The present disclosure is not limited to the above-described example embodiments, and can be appropriately modified without departing from the spirit. For example, one or more of the above-described components of each device may be omitted as appropriate. Also, for example, one or more of the steps of the above-described flowcharts may be omitted as appropriate. Also, an order of one or more of the steps in the flowcharts described above may be changed as appropriate.

Further, in the above-described second and third example embodiments, it is assumed that a graph or the like is displayed after the end of the breathing training. However, the information processing apparatus may generate a graph or the like, based on data acquired until that time of the breathing training during the breathing training, and display the graph or the like on the display device 30 in real time, and update the graph or the like over time.

The display system 100 and the display system 100a may include a plurality of imaging devices 20. In this case, the subject 90 is photographed by using the plurality of imaging devices 20. Accordingly, since the subject 90 can be photographed from a plurality of viewpoints, occurrence of blind spots of the subject 90 at the time of photographing can be suppressed. Therefore, the displacement amount and the like can be detected more accurately.

Further, the display system 100 may be achieved by a device in which two or more of the imaging device 20, the display device 30, and the information processing device 10 are integrally configured. For example, the subject 90 may perform breathing training by using one device (such as a smartphone) including the imaging device 20, the display device 30, and the information processing apparatus 10. This allows breathing training to be performed without special equipment. For example, the subject 90 can exercise breathing training at home or the like without hesitation. Similarly, the display system 100a may be also achieved by a device in which two or more devices are integrally configured.

Note that skeleton data may not be acquired in a device such as a smartphone. In this case, the subject 90 may perform an operation in such a way as to designate a chest region and an abdominal region in the self-photographed image 20g. In addition, when one apparatus including the imaging device 20, the display device 30, and the information processing apparatus 10 is used, breathing training may be performed by using an apparatus including the imaging device 20 that cannot acquire three-dimensional data. For example, by installing a device in the lateral direction of the subject 90 and photographing the subject 90, the displacement amounts of the chest portion 92 and the abdomen portion 94 of the subject 90 and the like can be detected. The apparatus that achieves the display system 100 may detect only the amount of displacement or the like during the breathing training, and may display an image of the displacement amount or the like after the completion of the training. The apparatus may output evaluation information in voice during the breathing training.

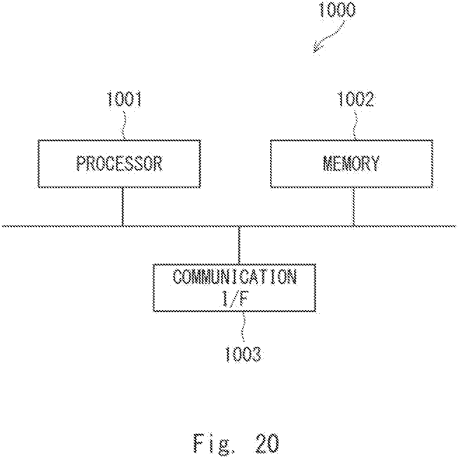

Each of the apparatuses according to the first to third example embodiments can have the following hardware configuration. FIG. 20 is a diagram illustrating an example of a hardware configuration to be included in the apparatus.

The apparatus 1000 illustrated in FIG. 20 includes a processor 1001, a memory 1002, and a communication interface 1003. The function of each device can be achieved by the processor 1001 reading a program stored in the memory 1002 and executing the program in cooperation with the communication interface 1003.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

The first to third example embodiments can be combined as desirable by one of ordinary skill in the art.

The whole or part of the exemplary example embodiments disclosed above can be described as, but not limited to, the following supplementary notes.

### (Supplementary Note 1)

An information processing apparatus including:
an input unit configured to input expiratory phase data being data of expiratory phase and inspiratory phase data being data of inspiratory phase, for each of abdominal waveform data indicating a breathing waveform of an abdomen of a subject performing breathing training and chest waveform data indicating a breathing waveform of a chest of the subject; and
a calculation unit configured to calculate a phase difference between the abdominal waveform data and the chest waveform data in each of the expiratory phase and the inspiratory phase.

### (Supplementary Note 2)

The information processing apparatus according to Supplementary note 1, wherein the calculation unit calculates, for the inspiratory phase data and the expiratory phase data, a distribution of the phase difference during a breathing cycle being normalized for each breathing cycle.

### (Supplementary Note 3)

The information processing apparatus according to Supplementary note 1 or 2, further including a display control unit configured to control a distribution of the phase difference to be displayed on a display device.

### (Supplementary Note 4)

The information processing apparatus according to Supplementary note 3, wherein the display control unit performs control of displaying in a different display mode according to a magnitude of the phase difference.

### (Supplementary Note 5)

The information processing apparatus according to Supplementary note 3 or 4, wherein the display control unit performs control of displaying, on the display device, a distribution of the phase difference for comparison, the distribution being calculated based on the past abdominal waveform data and the chest waveform data of the subject.

### (Supplementary Note 6)

The information processing apparatus according to Supplementary note 5, further including a selection unit configured to select the past abdominal waveform data and the past chest waveform data.

### (Supplementary Note 7)

The information processing apparatus according to Supplementary note 6, wherein the display control unit controls information indicating a start timing of the expiratory phase to be displayed in the distribution of the phase difference, and controls a target timing set for a start timing of the expiratory phase to be displayed in the distribution of the phase difference.

### (Supplementary Note 8)

The information processing apparatus according to any one of Supplementary notes 1 to 7, further including:
an input unit configured to input the abdominal waveform data and the chest waveform data; and
a division processing unit configured to divide each of the abdominal waveform data and the chest waveform data into the expiratory phase data and the inspiratory phase data, based on an average waveform data of the abdominal waveform data and the chest waveform data,
wherein the input unit inputs the expiratory phase data and the inspiratory phase data being divided by the division processing unit.

### (Supplementary Note 9)

The information processing apparatus according to any one of Supplementary notes 1 to 8, further including:
a waveform data input unit configured to input the abdominal waveform data and the chest waveform data; and
another display control unit configured to perform control of displaying, on a display device, a time-series displacement value of an abdomen that is indicated by the abdominal waveform data and a time-series displacement value of a chest that is indicated by the chest waveform data by potting one as a vertical axis and another as a horizontal axis.

### (Supplementary Note 10)

The information processing apparatus according to Supplementary note 9, wherein the another display control unit performs control of displaying markers representing the time-series displacement value of the abdomen and the time-series displacement value of the chest in such a way as to have a display mode different between a value close to a peak of exhalation and a value close to a peak of inspiration.

### (Supplementary Note 11)

An information processing apparatus including:
an input unit configured to input abdominal waveform data indicating a breathing waveform of an abdomen of a subject performing breathing training and chest waveform data indicating a breathing waveform of a chest of the subject; and
a display control unit configured to perform control of displaying, on a display device, a time-series displacement value of an abdomen that is indicated by the abdominal waveform data and a time-series displacement value of a chest that is indicated by the chest waveform data by plotting one as a vertical axis and another as a horizontal axis.

### (Supplementary Note 12)

The information processing apparatus according to Supplementary note 11, wherein the display control unit performs control of displaying markers representing the time-series displacement value of the abdomen and the time-series displacement value of the chest in such a way as to have a display mode different between a value close to a peak of exhalation and a value close to a peak of inspiration.

### (Supplementary Note 13)

An information processing method including:
inputting expiratory phase data being data of expiratory phase, and inspiratory phase data being data of inspiratory phase, for each of abdominal waveform data indicating a breathing waveform of an abdomen of a subject performing breathing training and chest waveform data indicating a breathing waveform of a chest of the subject; and
calculating a phase difference between the abdominal waveform data and the chest waveform data in each of the expiratory phase and the inspiratory phase.

### (Supplementary Note 14)

The information processing method according to Supplementary note 13, wherein the calculating includes calculating, for the inspiratory phase data and the expiratory phase data, a distribution of the phase difference during a breathing cycle normalized for each breathing cycle.

### (Supplementary Note 15)

The information processing method according to Supplementary note 13 or 14, further including performing control of displaying a distribution of the phase difference on a display device.

### (Supplementary Note 16)

The information processing method according to Supplementary note 15, wherein the performing control includes performing a control of displaying in a different display mode according to a magnitude of the phase difference.

### (Supplementary Note 17)

The information processing method according to Supplementary note 15 or 16, wherein the performing control includes performing control of displaying, on the display device, a distribution of the phase difference for comparison, the distribution being calculated based on past abdominal waveform data and past chest waveform data of the subject.

### (Supplementary Note 18)

The information processing method according to Supplementary note 17, further including selecting the past abdominal waveform data and the past chest waveform data.

### (Supplementary Note 19)

The information processing method according to Supplementary note 18, wherein the performing control includes performing control of displaying information indicating a start timing of the expiratory phase in the distribution of the phase difference, and performing control of displaying a target timing set for a start timing of the expiratory phase in the distribution of the phase difference.

### (Supplementary Note 20)

The information processing method according to any one of Supplementary notes 13 to 19, further including:
inputting the abdominal waveform data and the chest waveform data; and
dividing each of the abdominal waveform data and the chest waveform data into the expiratory phase data and the inspiratory phase data, based on average waveform data of the abdominal waveform data and the chest waveform data,
wherein the inputting includes inputting the expiratory phase data and the inspiratory phase data divided by the dividing.

### (Supplementary Note 21)

The information processing method according to any one of Supplementary notes 13 to 20, further including:
inputting the abdominal waveform data and the chest waveform data; and
performing another control of displaying, on the display device, a time-series displacement value of an abdomen that is indicated by the abdominal waveform data and a time-series displacement value of a chest that is indicated by the chest waveform data by plotting one as a vertical axis and another thereof as a horizontal axis.

### (Supplementary Note 22)

The information processing method according to Supplementary note 21, wherein the performing another control includes performing control to display markers representing the time-series displacement value of the abdomen and the time-series displacement value of the chest in such a way as to have a display mode different between a value close to a peak of exhalation and a value close to a peak of inspiration.

### (Supplementary Note 23)

An information processing method including:
inputting abdominal waveform data indicating a breathing waveform of an abdomen of a subject performing breathing training and chest waveform data indicating a breathing waveform of a chest of the subject; and
performing control of displaying, on a display device, a time-series displacement value of an abdomen that is indicated by the abdominal waveform data and a time-series displacement value of a chest that is indicated by the chest waveform data by plotting one as a vertical axis and another as a horizontal axis.

### (Supplementary Note 24)

The information processing method according to Supplementary note 23, wherein the performing control includes performing control of displaying markers representing the time-series displacement value of the abdomen and the time-series displacement value of the chest in such a way as to have a display mode different between a value close to a peak of exhalation and a value close to a peak of inspiration.

### (Supplementary Note 25)

A program causing a computer to execute information processing including:
inputting expiratory phase data being data of expiratory phase and inspiratory phase data being data of inspiratory phase for each of abdominal waveform data indicating a breathing waveform of an abdomen of a subject performing breathing training and chest waveform data indicating a breathing waveform of a chest of the subject; and
calculating a phase difference between the abdominal waveform data and the chest waveform data in each of the expiratory phase and the inspiratory phase.

### (Supplementary Note 26)

The program according to Supplementary note 25, wherein the calculating includes calculating, for the inspiratory phase data and the expiratory phase data, a distribution of the phase difference during a breathing cycle being normalized for each breathing cycle.

### (Supplementary Note 27)

The program according to Supplementary note 25 or 26, wherein the information processing includes performing control of displaying a distribution of the phase difference on a display device.

### (Supplementary Note 28)

The program according to Supplementary note 27, wherein the performing control includes performing control of displaying in a different display mode depending on a magnitude of the phase difference.

### (Supplementary Note 29)

The program according to Supplementary note 27 or 28, wherein the performing control includes performing control of displaying, on the display device, a distribution of the phase difference for comparison, the distribution being calculated based on past abdominal waveform data and past chest waveform data of the subject.

### (Supplementary Note 30)

The program according to Supplementary note 29, wherein the information processing includes selecting the past abdominal waveform data and the past chest waveform data.

### (Supplementary Note 31)

The program according to Supplementary note 30, wherein the performing control includes performing control of displaying information indicating a start timing of the expiratory phase in the distribution of the phase difference, and performing control of displaying a target timing set for a start timing of the expiratory phase in the distribution of the phase difference.

### (Supplementary Note 32)

The program according to any one of Supplementary notes 25 to 31, wherein
the information processing includes inputting the abdominal waveform data and the chest waveform data, and dividing each of the abdominal waveform data and the chest waveform data into the expiratory phase data and the inspiratory phase data, based on average waveform data of the abdominal waveform data and the chest waveform data, and
the inputting includes inputting the expiratory phase data and the inspiratory phase data being divided by the dividing.

### (Supplementary Note 33)

The program according to any one of Supplementary notes 25 to 32, wherein the information processing includes: inputting the abdominal waveform data and the chest waveform data; and performing another control of displaying, on a display device, a time-series displacement value of an abdomen that is indicated by the abdominal waveform data and a time-series displacement value of a chest that is indicated by the chest waveform data by plotting one as a vertical axis and another as a horizontal axis.

### (Supplementary Note 34)

The program according to Supplementary note 33, wherein the performing another control includes performing control of displaying markers representing the time-series displacement value of the abdomen and the time-series displacement value of the chest in such a way as to have a different display mode between a value close to a peak of exhalation and a value close to a peak of inspiration.

### (Supplementary Note 35)

A program causing a computer to execute information processing including:
inputting abdominal waveform data indicating a breathing waveform of an abdomen of a subject performing breathing training and chest waveform data indicating a breathing waveform of a chest of the subject; and
performing control of displaying, on a display device, a time-series displacement value of an abdomen that is indicated by the abdominal waveform data and a time-series displacement value of a chest that is indicated by the chest waveform data by plotting one as a vertical axis and another as a horizontal axis.

### (Supplementary Note 36)

The program according to Supplementary note 35, wherein the performing control includes performing control of displaying markers representing the time-series displacement value of the abdomen and the time-series displacement value of the chest in such a way as to have a display mode different between a value close to a peak of exhalation and a value close to a peak of inspiration.

According to the present disclosure, it is possible to provide an information processing apparatus, an information processing method, and a program capable of acquiring information indicating at which timing the synchronization between the abdomen and the chest of a subject performing breathing training is not achieved.

While the disclosure has been particularly shown and described with reference to example embodiments thereof, the disclosure is not limited to these example embodiments. It will be understood by those of ordinary skill in the art that various changes in form and details may be made therein without departing from the spirit and scope of the present disclosure as defined by the claims.

What is claimed is:

1. An information processing apparatus comprising
at least one memory storing instructions, and
at least one processor configured to execute the instructions to:
input expiratory phase data being data of expiratory phase and inspiratory phase data being data of inspiratory phase for each of abdominal waveform data indicating a breathing waveform of an abdomen of a subject performing breathing training and chest waveform data indicating a breathing waveform of a chest of the subject; and
calculate a phase difference between the abdominal waveform data and the chest waveform data in each of the expiratory phase and the inspiratory phase.

2. The information processing apparatus according to claim 1, wherein the calculating includes calculating, for the inspiratory phase data and the expiratory phase data, a distribution of the phase difference during a breathing cycle being normalized for each breathing cycle.

3. The information processing apparatus according to claim 1, wherein the at least one processor is to execute display control of displaying a distribution of the phase difference on a display device.

4. The information processing apparatus according to claim 3, wherein the display control includes control of displaying in a different display mode according to a magnitude of the phase difference.

5. The information processing apparatus according to claim 3, wherein the display control includes control of displaying, on the display device, a distribution of the phase difference for comparison, the distribution being calculated based on past abdominal waveform data and past chest waveform data of the subject.

6. The information processing apparatus according to claim 5, wherein the at least one processor is to select the past abdominal waveform data and the past chest waveform data.

7. The information processing apparatus according to claim 6, wherein the display control includes control of displaying information indicating a start timing of the expiratory phase in the distribution of the phase difference, and control of displaying a target timing set for a start timing of the expiratory phase in the distribution of the phase difference.

8. The information processing apparatus according to claim 1, wherein the at least one processor is to
input the abdominal waveform data and the chest waveform data, and
divide each of the abdominal waveform data and the chest waveform data into the expiratory phase data and the inspiratory phase data, based on average waveform data of the abdominal waveform data and the chest waveform data, and
the inputting includes inputting the expiratory phase data and the inspiratory phase data being divided by the dividing.

9. The information processing apparatus according to claim 1, wherein the at least one processor is to
input the abdominal waveform data and the chest waveform data, and
execute another display control of displaying, on a display device, a time-series displacement value of an abdomen that is indicated by the abdominal waveform data and a time-series displacement value of a chest that is indicated by the chest waveform data by plotting one as a vertical axis and another as a horizontal axis.

10. The information processing apparatus according to claim 9, wherein the another display control includes control of displaying markers representing the time-series displacement value of the abdomen and the time-series displacement value of the chest in such a way as to have a different display mode between a value close to a peak of exhalation and a value close to a peak of inspiration.

11. An information processing method comprising:
inputting expiratory phase data being data of expiratory phase and inspiratory phase data being data of expiratory phase, for each of abdominal waveform data indicating a breathing waveform of an abdomen of a subject performing breathing training and chest waveform data indicating a breathing waveform of a chest of the subject; and
calculating a phase difference between the abdominal waveform data and the chest waveform data in each of the expiratory phase and the inspiratory phase.

12. The information processing method according to claim 11, wherein the calculating includes calculating, for the inspiratory phase data and the expiratory phase data, a distribution of the phase difference during a breathing cycle normalized for each breathing cycle.

13. The information processing method according to claim 11, further comprising performing control of displaying a distribution of the phase difference on a display device.

14. The information processing method according to claim 13, wherein the performing control includes performing a control of displaying in a different display mode according to a magnitude of the phase difference.

15. The information processing method according to claim 13, wherein the performing control includes performing control of displaying, on the display device, a distribution of the phase difference for comparison, the distribution being calculated based on past abdominal waveform data and past chest waveform data of the subject.

16. A non-transitory computer readable medium storing a program that causes a computer to execute information processing comprising:
inputting expiratory phase data being data of expiratory phase and inspiratory phase data being data of inspiratory phase, for each of abdominal waveform data indicating a breathing waveform of an abdomen of a subject performing breathing training and chest waveform data indicating a breathing waveform of a chest of the subject; and
calculating a phase difference between the abdominal waveform data and the chest waveform data in each of the expiratory phase and the inspiratory phase.

17. The non-transitory computer readable medium according to claim 16, wherein the calculating includes calculating, for the inspiratory phase data and the expiratory phase data, a distribution of the phase difference during a breathing cycle normalized for each breathing cycle.

18. The non-transitory computer readable medium according to claim 16, the information processing further comprising performing control of displaying a distribution of the phase difference on a display device.

19. The non-transitory computer readable medium according to claim 18, wherein the performing control includes performing a control of displaying in a different display mode according to a magnitude of the phase difference.

20. The non-transitory computer readable medium according to claim 18, wherein the performing control includes performing control of displaying, on the display device, a distribution of the phase difference for comparison, the distribution being calculated based on past abdominal waveform data and past chest waveform data of the subject.
